(19) United States
(12) Patent Application Publication
(10) Pub. No.: US 2024/0270816 A1
(43) Pub. Date: Aug. 15, 2024

(54) FUSION PROTEIN FOR TREATING MYASTHENIA GRAVIS

(71) Applicant: TOLERANZIA AB, Göteborg (SE)

(72) Inventors: Jan-Olof ANDERSSON, Lindome (SE); Richard Ian CHRISTISON, Härryda (SE); Charlotte FRIBERT, Göteborg (SE); Björn LÖWENADLER, Askim (SE); Jennie SJÖHAMN, Västra Frölunda (SE)

(21) Appl. No.: 18/566,203

(22) PCT Filed: Jun. 7, 2022
(86) PCT No.: PCT/SE2022/050556

§ 371 Date: Dec. 1, 2023

Related U.S. Application Data
(60) Provisional application No. 63/197,722, filed on Jun. 7, 2021.

(30) Foreign Application Priority Data
Jun. 9, 2021 (SE) 2150726-4

(51) Int. Cl. C07K 14/705; A61K 38/00; A61P 21/04; C07K 1/04; C07K 14/435

(52) U.S. Cl. CPC C07K 14/70571; A61P 21/04; C07K 1/042; C07K 14/43504; A61K 38/00

(57) ABSTRACT

The invention relates to a method of producing a fusion protein between an extracellular domain of neuronal acetylcholine receptor subunit alpha 1 (nAChR&1) and a solubility enhancing peptide. The method comprises solubilizing inclusion bodies comprising the fusion protein in a solubilization solution having a pH of at least 11 to form solubilized fusion proteins. The method also comprises diluting the solubilized fusion protein in a refolding solution having a pH of no more than 9 to form a refolded monomeric form of the fusion protein.

### TECHNICAL FIELD

The present invention generally relates to fusion proteins useful in treatment of myasthenia gravis, and in particular to the production of such fusion proteins.

### BACKGROUND

Myasthenia gravis is a relatively rare autoimmune nerve-muscle disease that causes severe muscle weakness and, in many cases, a difficult life situation for the affected patient. The disease affects some 200 000 people in the EU and the US together and occurs in both sexes. It generally affects younger women (<40 years old) and older men (>60 years old).

Characteristic of the disease are fatigue and muscle weakness caused by an impaired transmission of nerve impulses to muscles. This is due to an immune attack directed against the acetylcholine receptor (AChR), which is the muscle relay station and the receiver of signals from the nerve. Destruction of AChR leads to defective neuromuscular conduction of electrical impulses and a subsequent muscle weakness, which can be very serious for the patient.

There are currently no treatments available that target the root cause of the disease. Rather, today, patients with myasthenia gravis are treated with symptom-relieving therapies, such as acetylcholinesterase inhibitors or immunosuppressive therapy, and, in many cases, thymectomy—a complicated surgical procedure, which removes the thymus. Common denominators of current treatments are that they have a limited effectiveness and can cause serious side effects. Therefore, there is a large unmet need for new and effective treatment options to offer patients with myasthenia gravis.

International applications WO 94/00148, WO 96/40777 and WO 98/50544 disclose recombinant fragments of the human AChR alpha subunit and their use for treatment of myasthenia gravis. International Journal of Biological Macromolecules (2014) 63: 210-217 discloses expression of human AChR extracellular domain mutants.

### SUMMARY

It is general objective to produce fusion proteins useful in treatment of myasthenia gravis.

This and other objectives are met by embodiments of the invention.

An aspect of the invention relates to a method of producing a fusion protein between an extracellular domain of nicotine acetylcholine receptor subunit alpha 1 (nAChRα1) and a solubility enhancing peptide. The method comprises solubilizing inclusion bodies comprising the fusion protein in a solubilization solution having a pH less than 11 and lacking any reducing agent to form solubilized fusion proteins. The method also comprises loading the solubilized fusion proteins onto an ion-exchange resin and eluting the loaded solubilized fusion proteins from the ion-exchange resin using an elution solution having a pH less than 11 and comprising a reducing agent to form a fusion protein eluate. The method further comprising adjusting a pH of the fusion protein eluate to at least 11 and diluting the pH-adjusted fusion protein eluate in a refolding solution having a pH of no more than 9 to form a refolded monomeric form of the fusion protein.

Another aspect of the invention relates to an isolated fusion protein between an extracellular domain of nAChRα1 as defined in SEQ ID NO: 3, in which amino acid residues 129 to 140 have been replaced by a solubility enhancing peptide. The asparagine residue 141 in SEQ ID NO: 3 is not glycosylated.

The invention also relates to the fusion protein of the invention for use as a medicament and for use in treatment or prophylaxis of myasthenia gravis.

The invention further defines a method for preventing, inhibiting or treating myasthenia gravis. The method comprises administering an effective amount of a fusion protein according to the invention to a subject in need thereof.

The present invention is capable of producing a fusion protein useful in treatment of myasthenia gravis, and in particular capable of obtaining the fusion protein in a monomeric form that is suitable to create or restore myasthenia gravis-specific tolerance in the immune system to the patient's own AChR proteins that the immune system incorrectly attacks in myasthenia gravis.

### BRIEF DESCRIPTION OF THE DRAWINGS

The embodiments, together with further objects and advantages thereof, may best be understood by making reference to the following description taken together with the accompanying drawings, in which:

FIG. 1 illustrates plasmid map of the pJ411 vector including protein 081 (TOL2).

FIG. 2 illustrates analytical SEC chromatogram of TOL2 after refolding. Monomeric TOL2 eluted at 16 ml. The major peak at 20 ml was non-protein compounds.

FIG. 3 illustrates chromatogram of AEX run. Absorbance at 280 nm (solid line) and conductivity (hatched line). TOL2 eluted at 40 mS (1200 ml).

FIG. 4 illustrates SEC chromatogram of TOL2. Monomeric TOL2 eluted at 1200 ml.

FIG. 5 illustrates analysis of final TOL2 sample. A) Analytical SEC chromatogram of the final TOL2 sample. Quantitation showed that 96% was monomeric TOL2. B) SDS-PAGE of TOL2 sample where quantitation show that 97% of the sample was monomeric TOL2.

FIG. 6 illustrates SDS-PAGE analysis of solubilization of TOL2 at different pHs.

FIG. 7 illustrates analysis of the solubilisation of TOL2 at pH 12.5 against 7 M urea at pH 8.0.

FIG. 8 illustrates non-reduced SDS-PAGE of refolded TOL2.

FIG. 9 illustrates SDS-PAGE for ion exchange fractions with reducing agent.

FIG. 10 illustrates SDS-PAGE for ion exchange fractions without reducing agent.

FIG. 11 illustrates SDS-PAGE for ion exchange fractions (M: Marker (SeeBlue Plus2 Pre-stained Protein Standard), 1: Input, 2: Flowthrough, 3: Wash—0% (with 100 mM cysteine), 4: Wash—0%, 5: Wash—7% of IEX 1 Buffer B, 6: Elution—25% of IEX 1 Buffer B, and 7: Strip—100% of IEX 1 Buffer B.

FIG. 12 illustrates intravenous administration of α1-ECDₘₜ results in a superior treatment effect compared to intranasal administration. EAMG rats received PBS via the intravenous route (N=6, empty circles) or 100 mg α1-ECDₘₜ in PBS via the intranasal (N=6, triangles) or the intravenous route (N=6, filled circles) on twelve consecutive days, from day 7 to day 18 of the experiment. (A) Average EAMG score (±SD) for each group; the grey bar indicates the treatment period. (B) Average percentage of body weight change (±SD). (C) CMAP decrement: each symbol corresponds to one rat and the bar shows the mean value. * Compares intravenous α1-ECDₘₜ and control.

FIG. 13 illustrates intravenous administration of α1-ECDₘₜ abrogates EAMG development in a dose and time dependent manner. (A-C) The data are compiled from 3 independent experiments. Rats were treated starting on day 7 with 5 mg (N=6, circles), 25 mg (N=14, triangles), 100 mg α1-ECDₘₜ (N=23, squares) or PBS (N=23, open circles). (D-F) The data are compiled from 2 independent experiments. Rats were treated starting on day 21 with 100 mg (N=21, circles), 500 mg α1-ECDₘₜ (N=7, triangles) or PBS (N=14, open circles). (G-I) The data are compiled from 2 independent experiments. Rats were treated starting on day 40 with 100 mg (N=14, circles), 500 mg (N=6, triangles), 1000 mg α1-ECDₘₜ (N=6, squares) or PBS (N=13, open circles). Grey bars, (in A, D, G) indicate the treatment period. The EAMG score (average±SEM), percentage of body weight change (average±SEM), and CMAP decrement were measured (for the later each symbol corresponds to one rat and the bar shows the mean value). In (A-C) #compares 5 μg vs 100 μg, +compares PBS vs 25 μg, and * compares PBS vs 100 μg. In (D-F)+compares PBS vs 100 μg and * compares PBS vs 500 μg. In (G-I) * compares PBS vs 1000 μg.

FIG. 14 illustrates serum levels of rat AChR and α1-ECDₘₜ antibodies following intravenous administration of α1-ECDₘₜ. (A) Serum samples were collected at the indicated time points after disease induction from rats treated with 100 mg α1-ECDₘₜ on day 7 (N=7, triangles), 500 mg on day 21 (N=7, circles), 1000 mg on day 40 (N=6, squares), or PBS (N=23, open circles), and were analyzed by RIPA for rat AChR antibodies. (B) The same serum samples as in (A) were assayed for α1-ECDₘₜ antibodies. (C) Healthy rats were injected with 500 mg α1-ECDₘₜ or PBS (N=10 for each group) during days 0-12 and days 50-62 as indicated by the gray bars, and serum samples collected at the indicated time points were analyzed for rat AChR and 1-ECDₘₜ antibodies (the lines show only α1-ECDₘₜ antibody titers, AChR antibodies were not detectable). In (A, B) * compares PBS vs Day 40, +compares Day 21 vs Day 40.

FIG. 15 illustrates EAMG treatment efficacy of α1-ECDₘₜ depends on dosing frequency. EAMG rats received α1-ECDₘₜ at different doses and frequencies starting from day 7 of the experiment and over 12 days: 100 mg every other day (N=7, circles; 600 mg in total), 200 mg every other day (N=7, triangles; 1200 mg in total), 200 mg every fourth day (N=7, squares; 600 mg in total), or 400 mg every fourth day (N=7, rhombus; 1200 mg in total). (A) Average EAMG scores (±SD); the grey bar indicates the treatment period and arrows the timing of each administration. A dotted line represents rats treated with 12×100 mg daily administrations for reference. (B) Average percentage of body weight change (±SD). (C) CMAP decrement: each symbol corresponds to one rat and the bar shows the mean value.

FIG. 16 illustrates EAMG treatment efficacy of α1-ECDₘₜ is superior to that of two different active treatments for MG. EAMG rats were treated with either 5 mg/kg methylprednisolone administered intraperitoneally (N=7), 18.5 mg/kg pyridostigmine administered orally (N=6), or 1000 mg α1-ECDₘₜ administered intravenously (N=6) on twelve consecutive days, starting at day 40 after disease induction. (A) Average EAMG scores (±SD); the grey bar indicates the treatment period. Circles correspond to α1-ECDₘₜ, triangles correspond to methylprednisolone and inverted triangles correspond to pyridostigmine. (B) Average percentage of body weight change (±SD). (C) CMAP decrement: each symbol corresponds to one rat and the bar shows the mean value. In (C, D) dotted lines represent untreated EAMG rats and healthy rats for reference. * Compares α1-ECDₘₜ and methylprednisolone.

FIG. 17 illustrates α1-ECDₘₜ has a short plasma half-life and is distributed to the liver, kidneys, and spleen upon intravenous administration. 100 μg ¹²⁵I-labelled α1-ECDₘₜ (106 cpm) were injected intravenously to healthy rats or EAMG rats on day 21 or day 40 after disease induction. (A) Blood was collected at various timepoints after intravenous injection and the residual ¹²⁵I-labelled & 1-ECDₘₜ was measured. 100% corresponds to the 106 cpm originally injected. (N=12 for each group). (B) The rats were sacrificed six hours after injection, selected organs were collected and measured. The value for each organ corresponds to recovered ¹²⁵I-labelled α1-ECDₘₜ minus the value contributed by residual ¹²⁵I-labelled α1-ECDₘₜ in the organ's estimated blood content. (N=12 for healthy and day 21 groups and N=10 for day 40 group).

FIG. 18 illustrates α1-ECDₘₜ and α1-ECDₘ display comparable treatment efficacies. EAMG rats received α1-ECDₘₜ or α1-ECDₘ by intravenous injection on twelve consecutive days from day 21 (100 mg; N=14 for α1-ECDₘₜ and N=7 for 1-ECDₘ) or day 40 (500 mg; N=6 for each group) of the experiment. (A) Average EAMG scores (±SD); the grey bar indicates the treatment period. Circles correspond to α1-ECDₘₜ and triangles correspond to α1-ECDₘ, while the dotted line represents untreated rats for reference. (B) Average percentage of body weight change (±SD). (C) CMAP decrement: each symbol corresponds to one rat and the bar shows the mean value. In (C) dotted lines represent untreated EAMG rats and healthy rats for reference

### DETAILED DESCRIPTION

The present invention generally relates to fusion proteins useful in treatment of myasthenia gravis, and in particular to the production of such fusion proteins.

There is currently an unmet medical need to find an effective treatment of myasthenia gravis. The present invention is based on the usage of a disease-specific fusion protein that is employed to create or restore myasthenia gravis-specific tolerance in the immune system to the patient's own AChR proteins that the immune system incorrectly attacks in myasthenia gravis. The fusion protein produced according to the invention acts as a myasthenia gravis tolerogen capable of restoring tolerance to the body's own AChR proteins that the immune system incorrectly attacks in myasthenia gravis.

AChR is an integral membrane protein that responds to the binding of the neurotransmitter acetylcholine. AChRs are typically classified as nicotinic acetylcholine receptors (nAChR) that are particularly responsive to nicotine and muscarinic acetylcholine receptors (mAChR) that are particularly responsive to muscarine. nAChRs are found in the central and peripheral nervous system, muscle, and many other tissues of human. At the neuromuscular junction they are the primary receptor in muscle for motor nerve-muscle communication that controls muscle contraction.

nAChR is made up of five subunits arranged symmetrically around a central pore. Each subunit comprises four transmembrane domains with both the N-terminal and the C-terminal located extracellularly. In humans and other vertebrates, nAChR are broadly classified into two subtypes muscle-type nAChR and neuronal-type nAChR. Muscle-type nAChR found at the neuromuscular junctions are either in an embryonic form, composed of α₁, β₁, γ, and δ subunits in a 2:1:1:1 ratio ((α₁)₂β₁γδ), or the adult form composed of α₁, β₁, δ, and εsubunits in a 2:1:1:1 ratio (α₁)₂β₁δε).

In humans, muscle-type nicotine acetylcholine receptor subunit alpha 1 (nAChRα1) is encoded by the CHRNA1 gene and is presented below and in SEQ ID NO: 1:

MEPWPLLLLF SLCSAGLVLG SEHETRLVAK LFKDYSSVVR
PVEDHRQVVE VTVGLQLIQL INVDEVNQIV TTNVRLKQGD
MVDLPRPSCV TLGVPLFSHL QNEQWVDYNL KWNPDDYGGV
KKIHIPSEKI WRPDLVLYNN ADGDFAIVKF TKVLLQYTGH
ITWTPPAIFK SYCEIIVTHF PFDEQNCSMK LGTWTYDGSV
VAINPESDQP DLSNFMESGE WVIKESRGWK HSVTYSCCPD
TPYLDITYHF VMQRLPLYFI VNVIIPCLLF SFLTGLVFYL
PTDSGEKMTL SISVLLSLTV FLLVIVELIP STSSAVPLIG
KYMLFTMVFV IASIIITVIV INTHHRSPST HVMPNWVRKV
FIDTIPNIMF FSTMKRPSRE KQDKKIFTED IDISDISGKP
GPPPMGFHSP LIKHPEVKSA IEGIKYIAET MKSDQESNNA
AAEWKYVAMV MDHILLGVFM LVCIIGTLAV FAGRLIELNQ QG

The extracellular domain of nAChRα1 corresponds to amino acids 21 to 255 of SEQ ID NO: 1 and is presented below and in SEQ ID NO: 2:

SEHETRLVAK LFKDYSSVVR PVEDHRQVVE VTVGLQLIQL
INVDEVNQIV TTNVRLKQGD MVDLPRPSCV TLGVPLFSHL
QNEQWVDYNL KWNPDDYGGV KKIHIPSEKI WRPDLVLYNN
ADGDFAIVKF TKVLLQYTGH ITWTPPAIFK SYCEIIVTHF
PFDEQNCSMK LGTWTYDGSV VAINPESDQP DLSNFMESGE
WVIKESRGWK HSVTYSCCPD TPYLDITYHF VMQRL

The fusion protein of the invention is based on the extracellular domain of nAChRα1 presented above and in SEQ ID NO: 2. In more detail, the fusion protein is based on amino acid residues 1-58, 84-235 in SEQ ID NO: 2 but in which twelve amino acids (marked in bold above) of a sequence motif denoted the Cys loop have been exchanged to improve solubility properties of the fusion protein and promote effective recombinant expression while retaining the native structure. The amino acid sequence omitting amino acid residues 59 to 83 in SEQ ID NO: 2 is presented below and in SEQ ID NO: 3:

SEHETRLVAK LFKDYSSVVR PVEDHRQVVE VTVGLQLIQL
INVDEVNQIV TTNVRLKQQW VDYNLKWNPD DYGGVKKIHI
PSEKIWRPDL VLYNNADGDF AIVKFTKVLL QYTGHITWTP
PAIFKSYCEI IVTHFPFDEQ NCSMKLGTWT YDGSVVAINP
ESDQPDLSNF MESGEWVIKE SRGWKHSVTY SCCPDTPYLD
ITYHFVMQRL

In a particular embodiment, twelve amino acids at position 129 to 140 in SEQ ID NO: 3 that is part of the sequence motif denoted the “Cys-loop” between Cys128 and Cys142 have been exchanged by twelve amino acids Asp132-Thr143 (DVSGVDTESGAT, SEQ ID NO: 4) from the homologous acetylcholine binding-protein (AChBP) of Lymnaea stagnalis. This amino acid sequence exchange has been made in order to improve solubility properties and promote effective recombinant expression while retaining the native structure. The fusion protein comprising the amino acid sequence Glu129-Gln140 in SEQ ID NO: 3 replaced by the amino acid sequence Asp132-Thr143 from L. stagnalis AChBP is presented below and in SEQ ID NO: 5:

SEHETRLVAK LFKDYSSVVR PVEDHRQVVE VTVGLQLIQL
INVDEVNQIV TTNVRLKQQW VDYNLKWNPD DYGGVKKIHI
PSEKIWRPDL VLYNNADGDF AIVKFTKVLL QYTGHITWTP
PAIFKSYCDV SGVDTESGAT NCSMKLGTWT YDGSVVAINP
ESDQPDLSNF MESGEWVIKE SRGWKHSVTY SCCPDTPYLD
ITYHFVMQRL

In an embodiment, a single N-terminal methionine (M) is added to the amino acid sequence in SEQ ID NO: 5 to enable bacterial expression of the fusion protein, such as in Escherichia coli. The resulting 211 amino acid fusion protein is presented below and in SEQ ID NO: 6:

MSEHETRLVA KLFKDYSSVV RPVEDHRQVV EVTVGLQLIQ
LINVDEVNQI VTTNVRLKQQ WVDYNLKWNP DDYGGVKKIH
IPSEKIWRPD LVLYNNADGD FAIVKFTKVL LQYTGHITWT
PPAIFKSYCD VSGVDTESGA TNCSMKLGTW TYDGSVVAIN
PESDQPDLSN FMESGEWVIK ESRGWKHSVT YSCCPDTPYL
DITYHFVMQR L

The fusion protein has four Cys residues forming two intramolecular disulfide bonds between Cys128-Cys142 and Cys192-Cys193, respectively with the amino acid numbering in accordance with SEQ ID NO: 3 and 5. The fusion protein has a molecular weight of 24194.47 Da and a theoretical isoelectric pH of 5.33.

In an embodiment, the asparagine residue 141 (Asp141) in SEQ ID NO: 3 (and 5) is not glycosylated. Asp141 forms, with the following two amino acids Cys142 and Ser142, a N-X-S sequon, which otherwise may be involved in N-linked glycosylation. However, in a preferred embodiment, no such N-linked glycosylation occurs on Asp141.

Recombinant production of the fusion protein is complicated by the formation of dimeric and higher multimeric forms of the fusion protein, including aggregates of the fusion proteins. Such dimeric and higher multimeric forms are generally not desired when using the fusion protein in treatment of myasthenia gravis. Hence, production of the fusion protein should result in the fusion protein in monomeric form.

An aspect of the invention relates to a method of producing a fusion protein between an extracellular domain of nAChRα1 and a solubility enhancing peptide. The method comprises solubilizing inclusion bodies comprising the fusion protein in a solubilization solution having a pH of less than 11 and lacking any reducing agent to form solubilized fusion proteins. The method also comprises loading the solubilized fusion proteins onto an ion-exchange resin and eluting the loaded solubilized fusion proteins form the ion-exchange resin using an elution solution having a pH less than 11 and comprising a reducing agent to form a fusion protein eluate. The method further comprises adjusting a pH of the fusion protein eluate to at least 11. The method additionally comprises diluting the pH-adjusted fusion protein eluate in a refolding solution having a pH of no more than 9 to form a refolded monomeric form of the fusion protein.

The fusion protein is effectively expressed and produced in host cells, preferably bacterial cells, such as E. coli cells, and accumulates in high quantities in intracellular inclusion bodies. The inventors have unexpectedly discovered that a significant drop in pH is required between solubilization of the inclusion bodies and refolding of the fusion protein in order to obtain the fusion protein in refolded monomeric form. Hence, performing the all the method steps in substantially the same pH, i.e., without any significant drop, results in accumulation of refolded fusion protein in dimeric and higher multimeric forms, including larger aggregates.

The fusion protein comprises multiple cysteine residues, i.e., four such cysteine residues in SEQ ID NO: and 6. Generally, a reducing agent is included in the solutions comprising proteins having such cysteine residues in order to prevent formation of undesired disulfide bonds within a protein molecule (intra-disulfide bonds) and/or between different protein molecules (inter-disulfide bonds) during the production process. For instance, such a reducing agent is generally included in a protein containing solution prior to loading the protein onto an ion-exchange resin for the purpose of purifying the protein by ion-exchange chromatography, such as anion-exchange chromatography.

Experimental data as presented herein, however, showed that the loading capacity of the ion-exchange resin with regard to the fusion protein was significantly lower when including a reducing agent during the loading step. Accordingly, omitting the reducing agent during the loading step significantly enhanced the loading capacity of the ion-exchange resin and thereby also significantly increased the yield of the fusion protein following anion-exchange chromatography. This was highly surprising and unexpected since one would presume the opposite, i.e., lower loading capacity and lower yield, due to the formation of aggregates of the fusion proteins due to the formation of inter-disulfide bonds when there is no reducing agent present in the solution.

In an embodiment, the method also comprises expressing the fusion protein in bacterial cells comprising an expression vector comprising a nucleotide sequence encoding the fusion protein under control of a promoter. In a particular embodiment, the bacterial cells are E. coli cells, such as the strain BL21(DE3) Star (Invitrogen). An illustrative, but non-limiting, example of an expression vector that could be used according to the invention is the expression vector pJexpress 411 (Atum). The nucleotide sequence encoding the fusion protein is under transcriptional control of a promoter. In a preferred embodiment, the promoter is an inducible promoter to enable control and timing of transcription of the nucleotide sequence encoding the fusion protein and thereby of production of the fusion protein. As an illustrative, but non-limiting, example, the promoter is an isopropyl β-d-1-thiogalactopyranoside (IPTG) inducible promoter, such as an IPTG-inducible T7 promoter.

In an embodiment, the method also comprises lysing the bacterial cells to form a lysate comprising the inclusion bodies and centrifuging the lysate to collect the inclusion bodies. In an embodiment, the inclusion bodies are also washed in at least one wash step.

The solubilization solution used according to the present invention has a basic pH, which in addition is preferably higher than the pH of the refolding solution. In an embodiment, the solubilization solution has a pH selected within an interval of from 9 to 10. In a preferred embodiment, the solubilization solution has a pH selected within an interval of from 9.2 to 9.8, and more preferably within an interval of from 9.4 to 9.6. Currently preferred pH value of the solubilization solution is about 9.5.

In an embodiment, the solubilization solution comprises urea, preferably at least 5 M urea. In a preferred embodiment, the solubilization solution comprises at least 6 M urea, and more preferably at least 7 M urea. A currently preferred concentration of urea in the solubilization solution is about 8 M.

In an embodiment, the solubilization solution may also comprise additional components, such as ethylenediaminetetraacetic acid (EDTA) used as metal chelating agent and glycine used to prevent pH decrease in the solubilization solution and to stabilize the fusion protein.

A currently preferred solubilization solution is 10 mM glycine, 8 M urea, 5 mM EDTA, PH 9.5. Hence, the solubilization solution lacks any reducing agent, such as cysteine.

The elution solution used to elute the fusion protein loaded onto the ion-exchange resin during anion-exchange chromatography has a basic pH, which in addition is preferably higher than the pH of the refolding solution. In an embodiment, the elution solution has a pH selected within an interval of from 9 to 10. In a preferred embodiment, the elution solution has a pH selected within an interval of from 9.2 to 9.8, and more preferably within an interval of from 9.4 to 9.6. Currently preferred pH value of the elution solution is about 9.5.

In an embodiment, the elution solution has a same pH as the solubilization solution.

The elution solution comprises a reducing agent. Various such reducing agents could be used according to invention including, but not limited to, cysteine, dithiothreitol (DTT) and 2-mercaptoethanol. A currently preferred reducing agent is cysteine.

In an embodiment, the elution solution comprises urea, preferably at least 5 M urea. In a preferred embodiment, the solubilization solution comprises at least 6 M urea, and more preferably at least 7 M urea. A currently preferred concentration of urea in the solubilization solution is about 8 M.

In an embodiment, the elution solution may also comprise additional components, such as EDTA and glycine.

An example of an elution solution is 10 mM glycine, 8 M urea, 5 mM EDTA, 10 mM cysteine, pH 9.5.

Another example of an elution solution is 10 mM glycine, 8 M urea, 5 mM EDTA, 10 mM cysteine, 1 M NaCl, pH 9.5. Further examples of elution solutions include mixtures of the two examples above to form an elution solution having a NaCl concentration between 0 M and 1 M. Hence, the elution solution comprises a reducing agent, i.e., cysteine.

The pH of the fusion protein eluate is adjusted to be at least 11. This pH adjustment can be performed by addition of NaOH, such as 1 M NaOH, until a desired basic pH is reached.

In an embodiment, the pH of the fusion protein eluate is adjusted to be within an interval of from 11 to 12. In a preferred embodiment, the solubilization solution has a pH selected within an interval of from 11.2 to 11.8, and more preferably within an interval of from 11.4 to 11.6. Currently preferred pH value of the solubilization solution is about 11.5 or 11.6, including a pH within an interval of from 11.5 and 11.6.

The refolding solution has a pH that is significantly lower than the pH of the pH-adjusted fusion protein eluate to achieve the pH drop promoting refolding of the fusion protein into monomeric form rather than multimeric forms of the fusion protein. The refolding solution preferably has a basic pH, i.e., a pH above 7. In an embodiment, the refolding solution has a pH selected within an interval of from 8 to 9. In a preferred embodiment, the refolding solution has a pH selected within an interval of from 8.4 to 8.8, and more preferably within an interval of from 8.5 to 8.7. Currently preferred pH values of the refolding solution are about 8.5 or about 8.6, including a pH within an interval of from about 8.5 to about 8.6.

The refolding of the fusion protein is preferably performed by diluting the pH-adjusted fusion protein eluate in the refolding solution. Experimental data have shown that correct and efficient refolding of the fusion protein is obtained when the fusion protein is present in a comparatively low concentration. Hence, the solubilized fusion protein is thereby diluted in the refolding solution to induce correct refolding of the fusion protein to obtain the refolded fusion protein in monomeric form.

In an embodiment, diluting the pH-adjusted fusion protein eluate comprises diluting the pH-adjusted fusion protein eluate in the refolding solution at a volume ratio of less than 0.1:1. The volume ratio X:Y as used herein is between the volume of the solubilized fusion protein (relative volume X) and the volume of the refolding solution (relative volume Y). In a preferred embodiment, diluting the solubilized fusion protein comprises diluting the solubilized fusion protein in the refolding solution at a volume ratio of less than 0.075:1 and more preferably less than 0.05:1, such as about 0.025:1.

As an illustrative example, one volume of the pH-adjusted fusion protein eluate is diluted in four volumes of refolding solution, such as by slowly adding the pH-adjusted fusion protein eluate to the refolding buffer over an extended period of time, such as at least 0.5 h, preferably at least 1 h, and more preferably at least 1.5 h, such as about 2 h. The diluted fusion protein is then preferably incubated over an extended period of time, such as at least 1 h, preferably at least 2 h, and more preferably at least 4 h, such as at least 5 h, at least 6, h, at least 7 h, at least 8 h, at least 9 h or at least 10 h or even longer, such as at least 12 h.

In an embodiment, the refolding solution comprises Tris-HCl to provide a buffering environment and keeping the pH of the refolding solution stable during the refolding. The refolding solution preferably comprises EDTA, reducing agent (cysteine), sucrose and glycerol.

A currently preferred refolding solution is 100 mM Tris-HCl, 5 mM EDTA, 4 mM cysteine, 400 mM sucrose, and 10% glycerol, pH 8.6.

In an embodiment, the method also comprises a purifying process comprising purifying the refolded monomeric form.

In an embodiment, the purifying process comprises purifying the refolded monomeric form of the fusion protein by ion exchange chromatography, preferably anion exchange chromatography. This purification step removes host cell impurities and other purities from the upstream culturing and cell lysing steps. In addition, the purifying process leads to a concentration of the refolded fusion protein by removal of water.

In an embodiment, the method comprises loading a chromatography column with an amount of the refolded monomeric form of the fusion protein that is below a maximum protein loading capacity of the chromatography column. Hence, in this embodiment, the chromatography column used in the ion exchange chromatography, preferably the anion exchange chromatography, is loaded below maximum protein loading capacity of the chromatography column. Experimental data indicates that using a sub-threshold fusion protein loading reduces the risk of eluting the fusion protein in higher multimeric forms.

Hence, the sub-threshold loading of the chromatography column contributes to obtaining the purified refolded fusion protein mainly in the monomeric form.

In an embodiment, the method comprises equilibrating a chromatography column with a buffer solution comprising poly(ethylene glycol)-poly(propylene glycol)-poly(ethylene glycol) triblock copolymer. Hence, in this embodiment, the chromatography column used in the ion exchange chromatography, preferably the anion exchange chromatography, is preferably equilibrated by poly(ethylene glycol)-poly(propylene glycol)-poly(ethylene glycol) triblock copolymers prior to loading the column with refolded protein. The poly(ethylene glycol)-poly(propylene glycol)-poly(ethylene glycol) triblock copolymer is preferably selected among PLURONIC® copolymers, such as PLURONIC® F127.

Equilibrating the chromatography column used in the ion exchange chromatography, preferably the anion exchange chromatography, increased the yield of the monomeric form of the fusion protein during the purifying process as compared to using a corresponding chromatography column without any poly(ethylene glycol)-poly(propylene glycol)-poly(ethylene glycol) triblock copolymer.

In an embodiment, the purifying process also comprises concentrating the purified refolded monomeric form of the fusion protein by tangential flow filtration (TFF). Such a TFF achieves an additional concentration of the fusion protein. In addition, TFF could be used to achieve a buffer shift of the solution, in which the fusion protein is dissolved.

In an embodiment, the purifying process further comprises purifying the concentrated refolded monomeric form of the fusion protein by size-exclusion chromatography (SEC). Such SEC achieves additional purification of the fusion protein by removing any impurities, such as host cell proteins, host cell nucleotide molecules and aggregates of the fusion protein.

In an embodiment, the purifying process comprises a second concentration of the purified refolded monomeric form of the fusion protein by tangential flow filtration following SEC.

The purifying process may optionally comprise one or multiple filtration steps, such as following the TFF and/or the SEC, using a filter, such as a 0.22 μm filter. Such an optional filtration step may also, or alternatively, be performed following solubilization of the inclusion bodies and prior to loading the solubilized fusion proteins onto the ion-exchange resin. The one or more optional filtration steps remove large particles and aggregates, which may otherwise interfere with the anion-exchange chromatography or SEC.

In an embodiment, the resulting concentrated fusion protein may be used as active pharmaceutical ingredient (API). Optionally, the fusion protein may be diluted to obtain a desired API concentration of the monomeric form of the fusion protein.

In an embodiment, the diluting step is performed at a temperature of no more than 10° C. In a preferred embodiment, the diluting step is performed at a temperature selected within an interval of from 4° C. to 10° C. In a preferred embodiment, the above described steps of the purifying process, or at least a portion of these steps, are preferably also conducted in a temperature of no more than 10° C.

The present invention also relates to a fusion protein between an extracellular domain of nAChRα1 as defined in SEQ ID NO: 3, in which amino acid residues 129 to 140 have been replaced by a solubility enhancing peptide. According to the invention, the asparagine residue 141 in SEQ ID NO: 3 is not glycosylated.

In a particular embodiment, the fusion protein comprises the extracellular domain of nAChRα1 as defined in SEQ ID NO: 3, in which amino acid residues 129 to 140 have been replaced by amino acid residues 132 to 143 from L. stagnalis AChBP as defined in SEQ ID NO: 4.

In an embodiment, the fusion protein comprises, preferably consists of, the amino acid sequence as defined in SEQ ID NO: 5 or 6.

In a preferred embodiment, the fusion protein consists of an N-terminal methionine residue followed by the extracellular domain of nAChRα1 as defined in SEQ ID NO: 3, in which amino acid residues 129 to 140 have been replaced by amino acid residues 132 to 143 from L. stagnalis AChBP as defined in SEQ ID NO: 4.

In a most preferred embodiment, the fusion protein consists of the amino acid sequence as defined in SEQ ID NO: 6.

The fusion protein is preferably in a monomeric form.

In an embodiment, the monomeric form of the fusion protein is preferably folded in the same way as the wild-type extracellular domain of nAChRα1, and more preferably in the same way as the extracellular domain of nAChRα1 is folded in the adult form of the muscle-type nAChR ((α₁)₂β₁δε).

In an embodiment, the fusion protein has a first disulfide bond between cysteine residue 128 in SEQ ID NO: 3 and 5 and cysteine residue 142 in SEQ ID NO: 3 and 5 and a second disulfide bond between cysteine residue 192 in SEQ ID NO: 3 and 5 and cysteine residue 193 in SEQ ID NO: 3 and 5. In an embodiment, the fusion protein has a molecular weight of about 24 kDa.

An embodiment also relates to a nucleotide sequence encoding a fusion protein according to the invention.

A nucleotide sequence, also referred to as nucleic acid sequence or nucleotide or nucleic acid molecule herein, refers to a polymer composed of nucleotides, such as ribonucleotides, deoxyribonucleotides, related naturally occurring structural variants, and/or synthetic non-naturally occurring analogs thereof, linked via phosphodiester bonds, related naturally occurring structural variants, and/or synthetic non-naturally occurring analogs thereof. Examples of such nucleotide sequences are deoxyribonucleic acid (DNA) sequences, ribonucleic acid (RNA) sequences, complementary DNA (cDNA) sequences and messenger RNA (mRNA) sequences.

Various nucleotide sequences are possible to encode a single fusion protein due to the degeneracy of the genetic code and a single amino acid may be coded for by more than one codon. An example of a nucleotide sequence encoding a fusion protein of the invention is shown in SEQ ID NO: 7. The embodiments are, however, not limited to this particular example of nucleotide sequences but also include variants thereof having a different nucleotide sequence than the one shown in any of SEQ ID NO: 7 but still, due to the degeneracy of the genetic code, encodes the same fusion protein as the nucleotide sequence shown in the any of SEQ ID NO: 7. For instance, such a different nucleotide sequence may be a codon optimized version of the nucleotide sequence. A codon optimized version of a nucleotide sequence refers to a nucleotide sequence where the codons have been optimized with regard to a particular cell used to express the polypeptide from the nucleotide sequence. Generally, not all transfer RNAs (tRNAs) are expressed equally or at the same level across species. Codon optimization of a nucleotide sequence thereby involves changing codons to match the most prevalent tRNAs, i.e., to change a codon recognized by a low prevalent tRNA with a synonymous codon recognized by a tRNA that is comparatively more prevalent in the given cell. This way the messenger RNA (mRNA) from the codon optimized nucleotide sequence will be more efficiently translated. The codon and the synonymous codon encode the same amino acid.

An embodiment relates to a vector, in particular an expression vector, comprising a nucleotide sequence of the invention under transcription control of a promoter.

As used herein, the term promoter refers to a nucleic acid sequence which has functions to control the transcription of a polypeptide-coding nucleotide sequence (gene), and is located upstream with respect to the direction of transcription of the transcription initiation site of the polypeptide-coding nucleotide sequence. Suitable promoters in this context include both constitutive and inducible natural promoters as well as engineered promoters, which are well known to the person skilled in the art.

The promoter is preferably selected based on the type of host cell that will be used to produce the polypeptide encoded by the nucleic sequence included in the expression vector under transcriptional control of the promoter.

Illustrative, but non-limiting, examples of a promoter that could be used for expression of the nucleotide sequence in a bacterial cell, such as an E. coli cell, include the lac promoter, the T7 promoter, the Tac promoter, and the trp promoter. Illustrative, but non-limiting, examples of a promoter that could be used for expression of the nucleotide sequence in a yeast cell, such as a Saccharomyces cerevisiae cell, a Pichia pastoris cell, a Yarrowia lipolytica cell or a Kluyveromyces lactis cell, include the AOX1 promoter, the LAC4 promoter, the GAL1 promoter, the GAL2 promoter, the GAL7 promoter, the GAL10 promoter, the TEF1 promoter, the PGK1 promoter, the PDC1 promoter, the ENO1 promoter, the TPI1 promoter, and the G3P promoter. An illustrative, but non-limiting, example of a promoter that could be used for expression of the nucleotide sequence in insect cells, such as Lepidoptera cells, is the polyhedrin promoter of baculovirus. Illustrative, but non-limiting, examples of a promoter that could be used for expression of the nucleotide sequence in a mammalian, such as human, cell include the CMV promoter, the SV40 promoter and the EF-1 promoter.

A nucleotide sequence is under transcription control of a promoter when the promoter is located relative to the nucleotide sequence to control transcription of the nucleotide sequence into an mRNA sequence.

The vector is preferably an expression vector, i.e., a vector comprising at least one nucleotide molecule comprising a fusion protein-coding nucleotide sequence that can be expressed, such as transcribed and translated, in a host cell comprising the expression vector. The expression vector is in an embodiment selected among DNA molecules, RNA molecules, plasmids, episomal plasmids and virus vectors. Illustrative, but non-limiting examples, of virus vectors include a lentiviral vector, an adenoviral vector, an adeno-associated viral vector, a retroviral vector, a baculovirus and a hybrid vector.

A related embodiment defines a host cell comprising an expression vector of the invention. The host cell can then be used to express the fusion protein-coding nucleotide sequence in the expression vector to thereby produce a polynucleotide of the invention. The host cell could, for instance, be a bacterial cell, such an E. coli cell, a yeast cell, such as a S. cerevisiae cell, a P. pastoris cell, a Y. lipolytica cell or a K. lactis cell, an insect cell, such as a Lepidoptera cell, or a mammalian cell, such as a human cell.

The fusion proteins of the invention can be used as tolerogens as they are capable of restoring tolerance to the body's own proteins that the immune system incorrectly attacks in autoimmune disease.

The present invention also relates to a fusion protein of the invention for use as a medicament and for use in treatment or prophylaxis of myasthenia gravis. A related aspect of the invention defines the use of the fusion protein for the manufacture of a medicament for treatment or prophylaxis of myasthenia gravis.

Treatment or treating as used herein means an approach for obtaining beneficial or desired results, including clinical results. Beneficial or desired clinical results could include, for instance, alleviation or amelioration of one or more symptoms or conditions, diminishment of extent of myasthenia gravis, stabilized state of myasthenia gravis, i.e., prevent worsening, preventing spread of myasthenia gravis, delay or slowing of disease progression, amelioration or palliation of the disease state, diminishment of the reoccurrence of myasthenia gravis, and remission. Treatment or treating may also prolong survival as compared to expected survival if not receiving any treatment.

Preventing or prophylaxis as used herein means an approach in which a risk of developing myasthenia gravis is reduced or prevented, including prolonging or delaying myasthenia gravis development. For instance, a patient predisposed to develop a disease, such as due to genetic or hereditary predisposition, could benefit for administration of the polypeptide or a pharmaceutical composition comprising the polypeptide to prevent, reduce the risk of, delaying and/or slowing development of myasthenia gravis.

The fusion protein may be administered to a subject or patient in need thereof in the form of a pharmaceutical composition comprising the fusion protein of the invention.

The pharmaceutical composition may additionally comprise one or more pharmaceutically acceptable additives including, but not limited to, carriers, vehicles, diluents, adjuvant, aroma, preservatives and/or excipients. Non-limiting examples of a pharmaceutically acceptable carrier or vehicle is an injection solution, such as saline or a buffered injection solution.

The pharmaceutical composition may, for instance, be in the form of a tablet, a capsule, powder, nanoparticles, a solution, such as an injection solution, a transdermal patch or a suppository.

A currently preferred pharmaceutical composition is an aqueous injection solution comprising the fusion protein at 3.0 mg/ml in 40 mM Tris, 150 mM NaCl, pH 8.5.

The pharmaceutical composition preferable comprises an effective amount of the fusion protein. As used herein, effective amount indicates an amount effective, at dosages and for periods of time necessary to achieve a desired result. Effective amounts may vary according to factors, such as the disease state, age, sex, weight of the patient.

The patient is preferably a human patient.

A further aspect of the invention relates to a method for preventing, inhibiting or treating myasthenia gravis. The method comprises administering an effective amount of a fusion protein or pharmaceutical composition of the invention to a subject in need thereof.

Alternatively, or in addition, an effective amount of a nucleotide sequence and/or expression vector of the embodiment, or a pharmaceutical composition comprising the nucleotide sequence and/or expression vector, is administered to the subject in need thereof.

The fusion protein or the pharmaceutical composition may be administered to the subject according to various routes including, for instance, intravenous, subcutaneous, intraperitoneal, intramuscular, topical, nasal, buccal, sublingual or oral administration, or administration via the respiratory tract, such as, in the form of an aerosol or an air-suspended fine powder.

### EXAMPLES

### Example 1—Construction and Expression of TOL2

This examples describes generation of an expression system in Escherichia coli BL21Star(DE3) under control of an inducible T7 promoter capable of expressing TOL2 (SEQ ID NO: 6) in high yields.

### Expression System Generation

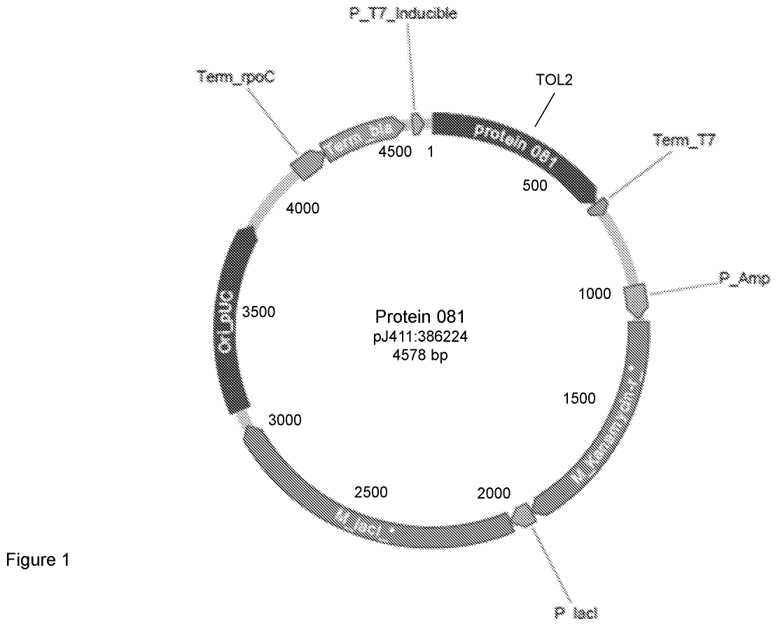

The nucleotide sequence (SEQ ID NO: 7) encoding TOL2 was cloned into the expression vector pJ 411 (FIG. 1). E. coli BL21 Star (DE3) cells were transformed with the plasmid pJexpress 411-TOL2 by heat shock procedure following manufacturer's protocol (Invitrogen: 30 min 4° C., 30 sec 42° C.). Two different amounts of DNAs (5 and 10 ng) were used. Transformed cells were spread on LB agar plates containing kanamycin (50 μg/mL). Five colonies were selected from the first plate and re-streaked in a new plate.

### Expression System Characterization

Five different colonies from the transformation, each of them from a different plate, were used to inoculate 25 mL fresh animal free LB medium with kanamycin (50 μg/mL) and the culture was maintained overnight (O/N) at 37±1° C. and 225±10 rpm.

From this initial culture, 15 mL were centrifuged (7300 g during 5 min at room temperature (RT, 20-25° C.) and pellets were re-suspended in 5 mL of fresh LB medium with 20% glycerol but no antibiotics. These 5 mL were distributed in five cryovials to create pre-cell banking. This mini cell bank was stored at −80±10° C. under continuous temperature monitoring. From the overnight culture, 3 mL were used to perform the following assays to fully characterize the expression system.

### Growth Parameters Evaluation

In order to study the ability of the expression system to grow, 1 mL from the O/N culture of each of the five colonies was used to inoculate 25 mL of fresh LB medium without antibiotics. The culture was maintained at 37±1° C. and 250±10 rpm. The OD₆₀₀ was monitored every hour during 12 h using a UV-Vis spectrophotometer; some samples were taken the next morning as well. A sample was taken at the end of the growth curve as a non-induced sample.

### DNA Analysis

From the above mentioned O/N culture, 1.5 mL of culture of each of the five colonies was used for DNA extraction following QIAprep Spin Miniprep kit manufacturer's protocol (QIAGEN). Part of the purified DNA corresponding to the coding protein region was sequenced.

### Protein Expression Analysis

An induction assay was performed to study protein expression. A volume of 0.2 mL of one vial of the pre-cell banking of the five colonies was used to inoculate 50 ml of fresh LB medium without antibiotics. The culture was maintained at 37±1° C. and 225±10 rpm and OD₆₀₀ was monitored until it reached 1±0.2. At that moment, 1 mL of culture was taken as a pre-induction sample (TO) and then, the culture was induced by the addition of 1 mM of IPTG. After the induction, the 50 mL were split and half of it was maintained at 37° C. and half of the material was put at 30° C. in a second incubator. Four hours later, a 3 ml sample was taken as post-induction sample (T1). Both TO and T1 samples were centrifuged (7300 g during 5 min at RT) and the pellets were collected and re-suspended in PBS normalizing the solution to OD₆₀₀=10. Once the pellets were re-suspended, 1 mL of T1 sample was sonicated (six pulses of 30 seconds each, with a stop of 30 seconds between pulses; 500 W, 20 KHz) obtaining the total extract (TE). From TE sample, 500 μl were centrifuged (7300 g during 5 min at RT) in order to obtain the insoluble fraction (IF, pellet) and the soluble fraction (SF, supernatant). Apart from TO, the rest of the samples were duplicated, one of the samples was from the experiment at 37±1° C. and the other from the experiment at 30±1° C.

All samples (TO, TE, IF, SF and non-induced) were analyzed by SDS-PAGE in order to determine, in which fraction, i.e., the soluble or the insoluble one, the TOL2 was expressed.

To confirm the identity with an affinity method, the same samples were loaded into another gel and this gel was transferred to a nitrocellulose membrane. A Western blot (WB) was done with a rabbit anti-TOL2 antibody following the protocol recommended (1:3000 for the primary antibody and 1:8000 for the secondary anti-rabbit antibody)

### RCB Generation and Characterization

For the generation of general cell line (RCB), 300 UL of one vial of the pre-cell banking of the selected colony was used. Briefly, one vial of the mini bank was used to inoculate 100 ml of Phytone TB medium and OD₆₀₀ was monitored until the culture reached mid-exponential phase (OD₆₀₀=0.8±0.2). At that moment, the culture was harvested and centrifuged (8000 g, during 5 min at 4° C.) and the pellet was re-suspended in 50 mL of Phytone TB+20% glycerol, and was dispensed into 50×1 mL cryovials. The vials were stored at −80±10° C.

The components of the medium are shown in Table 1.

TABLE 1
Components of Phytone TB medium

| Component | Amount per 1 L | |
|---|---|---|
| Phytone | 12 | g |
| Yeast extract | 24 | g |
| KH₂PO₄ | 2.31 | g |
| K₂HPO₄ | 12.54 | g |
| Glycerol | 3.17 | mL |

The cryopreservation medium used was the same recipe but including an extra 20% of glycerol.

### Plasmid Identity

In order to confirm the sequence of the DNA fragment corresponding to the coding protein region, isolated colonies from RCB in a TSA plate were sequenced.

### Results

### Vector Transformation

More than 50 colonies were obtained from each of the transformation (with 5 and 10 ng of plasmid). The plates chosen for the selection of the colonies were the one with 10 ng and 50 μL of material spread. Fibe colonies were fully characterized.

### Expression System Characterization

The growth capacity was the same for all E. coli BL21Star(DE3)-TOL2 clones reaching maximum OD₆₀₀ of 7 after 11 h of culture. The expression system E. coli BI21Star(DE3)-pJ411 TOL2 was able to grow in a rich media, such as LB medium, reaching high values of OD₆₀₀ in a few hours.

### Protein Expression Analysis

After the induction assay, the samples collected from the five different colonies and normalized to OD₆₀₀ were analyzed in a 12% SDS-PAGE Coomassie gel. The results showed a clear overexpresion of one protein in the induced sample (TE, lane 4) with a molecular weight (Mw) of about 25 kDa (13.86 kDa), similar to the Mw of the reference material (loaded in lane 2). The non-induced sample (lane 10) maintained over night showed an overexpressed protein of that size.

The band of about 25 kDa also appeared in the insoluble fraction and was not present in the soluble fraction. This means that the protein was present inside the cells forming inclusion bodies.

The percentage of the overexpressed protein in the total extract sample (TE, lanes 4 and 7) was calculated by densitometry of the bands within that lane with the software of Imagelab (Syngene). The percentage of the band for each colony at each temperature is shown in Table 2.

TABLE 2
Percentage of the band of interest calculated by densitometry

| Colony | % expression at 37° C. | % expression at 30° C. |
|---|---|---|
| 1 | 33.9 | 25.9 |
| 2 | 38.6 | 27.8 |
| 3 | 35.1 | 28.9 |
| 4 | 29.8 | 24.8 |
| 5 | 22.1 | 19.2 |

From these results two observations were obtain; colonies 2 and 3 showed the higher expression level and the expression is higher at 37° C. than at 30° C.

SDS-PAGE result showed an overexpression of the protein with a similar Mw to the monomer of TOL2. In order to confirm the identity of the band a WB was performed using an anti-TOL2 antibody. A single band in the TE and IF samples was observed. The single band had a size of about 25 kDa. It was considered that the WB gave enough information about the identity of the protein of interest since a clear band of the expected Mw was observed.

### Plasmid Identity

Isolated colonies were subject to plasmid identity analysis. The DNA sequence of the region coding for the protein of interest was 100% identical compared with the original sequence. The manufacture of the RCB did not affect the DNA, preserving the original sequence.

### Example 2—Production and Purification of Monomeric TOL2

This example describes a process of producing and purifying monomeric TOL2.

### Preparation of Pre-Inoculum

In the biosafety cabinet, 200 ml of complete inoculum medium (0.88 kg/L base medium (2.272 g/L citric acid, 6.817 g/L KH₂PO₄, 11.361 g/L K₂HPO₄, 6.817 g/L Na₂HPO₄, 4.543 g/L (NH₄)₂SO₄) 103.10 g/L medium component (100.00 g/L D(+)-glucose monohydrate) and 20.00 g/L trace salts (2.160 g/L HCl 37%, 1.00 g/L FeCl₃·6H₂O, 0.600 g/L MnSO₄·H₂O, 0.200 g/L ZnCl₂, 0.150 g/L Na₂MoO₄·2H₂O, 0.050 g/L H₃BO₃, 0.250 g/L CoCl₂·6H₂O, 1.00 g/L CaCl₂·2H₂O, 0.250 g/L CuSO₄·5H₂O, 50.00 g/L MgSO₄·7H₂O) was transferred to a 500 ml Erlenmeyer flask. One vial from Example 1 comprising E. coli BL21Star(DE3)-TOL2 was thawed using the Multitron Incubator at 37° C. during 5 minutes. The content of the thawed vial was homogenized in the biosafety cabinet and 1 mL from the vial was added to 500 ml Erlenmeyer flask. Further, 100 ml of the inoculated media was transferred to a 500 mL Erlenmeyer flask. Thereafter, the culture was incubated at 37° C. and 230 rpm inside the Multitron Incubator until desired OD₆₀₀ of 1.440 AU was reached after 4 h and 35 min.

### Inoculum Expansion

In the biosafety cabinet, 800 ml of complete inoculum medium was transferred to a 2 L bottle. Afterwards, 5 ml of pre-inoculum culture was transferred to the bottle. Three 1 L Erlenmeyer flasks were aliquoted with 200 ml of the culture from the 2 L bottle. The three Erlenmeyer flasks were incubated at 37° C. and 230 rpm inside the Multitron Incubator until desired OD₆₀₀ of 2.900 AU was reached after 6 h 18 min. The contents of the three Erlenmeyer flasks were transferred to an autoclaved 1 L bottle identified as and shaken gently. 1 ml sample was measured OD₆₀₀ (3.230 AU) and the bioreactor was inoculated with 540 ml culture from 1 L bottle.

### Inoculation of Bioreactor

During bioreactor preparation, 79.3 L of F-100 medium (50 g/L glycerol, 0.88 kg/L base medium (2.272 g/L citric acid, 6.817 g/L KH₂PO₄, 11.361 g/L K₂HPO₄, 6.817 g/L Na₂HPO₄, 4.543 g/L (NH₄)₂SO₄)) was added to a F100 fermenter/bioreactor, followed by 200 ml of anti-foam (simethicone 9%). When the fermenter medium temperature reached 22° C., 1.6 L of trace salts (2.160 g/L HCl 37%, 1.00 g/L FeCl₃·6H₂O, 0.600 g/L MnSO₄·H₂O, 0.200 g/L ZnCl₂, 0.150 g/L Na₂MoO₄·2H₂O, 0.050 g/L H₃BO₃, 0.250 g/L CoCl₂·6H₂O, 1.00 g/L CaCl₂):2H₂O, 0.250 g/L CuSO₄·5H₂O, 50.00 g/L MgSO₄·7H₂O) was transferred to the culture medium. The bioreactor was inoculated with 540 mL of the inoculum giving a theoretical OD₆₀₀ of 0.0218 AU (0.020±0.004 AU).

The feed (120.00 g/L yeast extract, 5 g/L MgSO₄·7H₂O, 5 g/L (NH₄)₂SO₄, 700 g/L glycerol, 24.50 g/L simethicone 9%) was started at 13 hrs 19 min after inoculation with a flow rate of 28.2 mL/min. The feed ended 8 hrs 22 min after its start. The induction phase was started 20 minutes after feed start by loading 0.202 L of 200 mM IPTG. The induction phase lasted 8 hours, and optical density (OD₆₀₀) at the end of induction was 188.50 AU. Once the induction phase was completed, the feed pump stopped, and temperature was adjusted to 5° C. in order to start the cooling phase. After that, acid, base and antifoam controls were deactivated, and the stirrer speed was reduced by 50 rpm tranches until reaching a set point of 100 rpm. After 1.7 hrs, the bioreactor reached 12° C., and the process continued with the biomass harvest.

### Biomass Harvest

When the temperature of the fermentation product reached 10° C., the product transfer line was connected to a Westfalia centrifuge inlet. The centrifugation of the biomass harvest was performed using the following operating parameters: 11650 rpm centrifuge speed; 5.0% feed flow rate; 3.0 bar backpressure of supernatant line; 2.4 bar seal pressure; 0.4 min of discharge interval (5 partial discharges followed by a total discharge). The harvesting process lasted 1 h 30 min and 48.165 kg of biomass was collected into a storage tank.

Re-suspension buffer (50 mM Tris-Base, 5 mM EDTA, pH 8.0) was added to the storage tank with biomass to reach a total resuspension volume of 100 L. The re-suspended biomass was stirred at speed of 150 rpm for 29 minutes. The temperature during the resuspension was kept at 8° C. (≤10° C.).

### Cell Lysis

Two passes of lysis were performed using a GEA Panther cell disrupter. Temperature of the re-suspended biomass was 8° C. when lysis run 1 started. After 72 minutes with a working pressure of 600 bar and a feed pressure of 2.4 bar, the process ended, and 100 L of the lysate run 1 volume was collected into a 100 L Flexel liner bag in a tank. For the second run, a 200 L tank with 200 L Flexel bag installed in it was used to collect w the lysated product of the process. The process started with a temperature of 10° C., a working pressure of 640 bar, a feed pressure of 2.4 bar, and lasted for 78 minutes. After the lysate run 2, a total volume of lysate product of 100 L was collected.

Inclusion bodies were collected from the lysate product by centrifugation using a CEPA Z61 centrifuge at 16000 rpm at a temperature of 4° C. to form a centrifuged pellet.

### Inclusion Body Washes

Centrifuged pellet was re-suspended with 78.040 L of first wash buffer (50 mM Tris, 5 mM EDTA, 1 M NaCl, pH 8) and stirred at speed of 146 rpm for 30 minutes. The temperature during the wash was kept at 9° C. (≤10° C.). The washed inclusion bodies were centrifuged using CEPA centrifuge at 16040 rpm speed with 24 L/h feed flow during 4 hours and 35 minutes. Once the product was centrifuged, the equipment was fed with 7 L of the first wash buffer in order to drain the product from centrifugation system.

In a second wash step, the pellet of inclusion bodies was re-suspended with 5.020 L of second wash buffer (50 mM Tris-HCl 50, 5 mM EDTA, 0.25% DOC, pH 8) and stirred at speed of 150 rpm for 28 minutes. The temperature during the wash was kept at 9° C. (≤10° C.). The washed inclusion bodies were centrifuged using CEPA centrifuge at 16050 rpm speed with 9.5 L/h feed flow during 3 hours and 10 minutes. Once the product was centrifuged, the equipment was fed with 7 L of the second wash buffer in order to drain the product from centrifugation system.

In a third wash step, the pellet of inclusion bodies was re-suspended with 4.941 L of the second wash buffer (50 mM Tris-HCl, 5 mM EDTA, 0.25% DOC, pH 8) and stirred at speed of 150 rpm for 30 minutes. The temperature during the wash was kept at 9° C. (≤10° C.). The washed inclusion bodies were centrifuged using CEPA centrifuge at 16000 rpm speed with 9.0 L/h feed flow during 4 hours and 27 minutes. Once the product was centrifuged, the equipment was fed with 7 L of the second wash buffer in order to drain the product from centrifugation system.

In a fourth wash step, the pellet of inclusion bodies was re-suspended with 44.24 L of third wash bufferF (50 mM Tris-Base, 5 mM EDTA, pH 8.0) and stirred at speed of 150 rpm for 30 minutes. The temperature during the wash was kept at 9° C. (≤10° C.). The washed inclusion bodies were centrifuged using CEPA centrifuge at 16030 rpm speed with 24.0 L/h feed flow during 3 hours and 25 minutes. The heavy phase (inclusion bodies) was collected. Once the product was centrifuged, the equipment was fed with 7 L of the third wash buffer in order to drain the product from centrifugation system. The total weight of inclusion bodies obtained from centrifugation was 3.484 kg.

### Solubilization

TOL2 inclusion bodies (about 7 g) were re-suspended in 250 ml solubilization buffer (10 mM Na₂PO₄, 8 M urea, 5 mM EDTA, 100 mM cysteine, pH 11.6). The solution was stirred at room temperature and sonicated 4×2 min total time (15 s on, 15 s off, 70% amplitude) to re-suspend the inclusion bodies completely. The mixture was then stirred for 2 h at room temperature. Unsolubilized material was pelleted by centrifugation (38 000 g, 1.5 h, 4° C.). The solubilization suspension sample was kept at 4° C. until refolding.

### Refolding

10 L refolding buffer (100 mM Tris-HCl, 5 mM EDTA, 4 mM cysteine, 400 mM sucrose, 10% glycerol, pH 8.6) was equilibrated at 5° C. overnight and the solubilization suspension sample was added slowly over night (ca 10 h) in a continuous mode with stirring.

### Anion Exchange Chromatography (AEX)

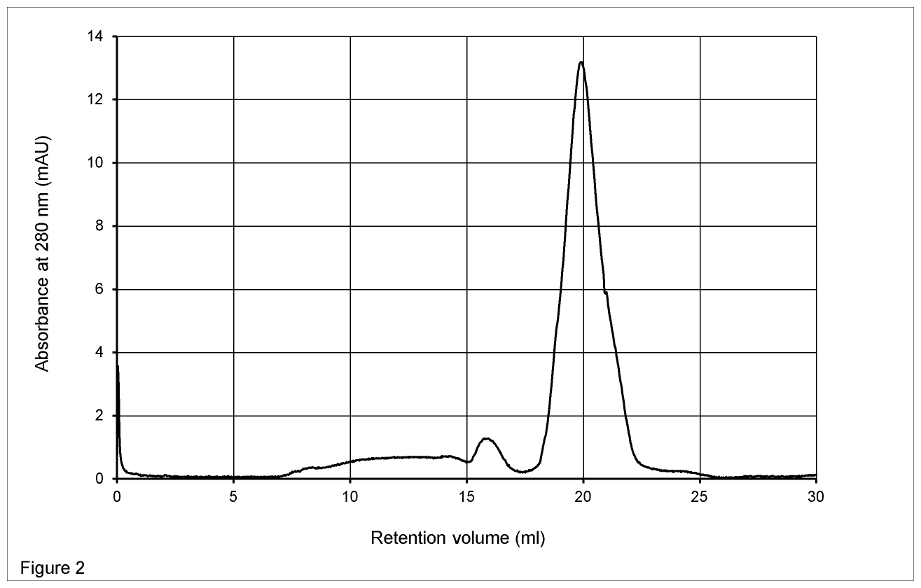

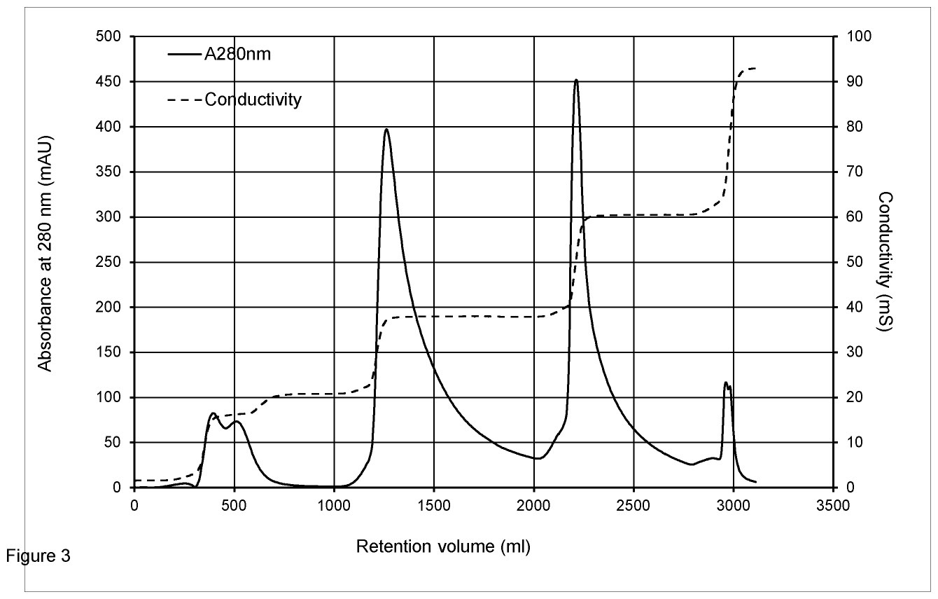

Refolded TOL2 was analyzed with analytical size exclusion chromatography (SEC) (Superdex200 10/300), see FIG. 2. Refolded TOL2 was loaded onto a XK 50/30 packed with QSepharose FF (final bed volume 360 ml) equilibrated with Buffer A (40 mM Tris, 0.02% PLURONIC® F127, pH 8.5). The bound protein was washed with the same buffer containing 180 mM NaCl. Elution of TOL2 was made with stepwise increases of NaCl to 350 mM and 600 mM (FIG. 3).

Investigations of how the column diameter impacted the recovery of monomeric TOL2 showed that a larger diameter on the AEX column increased the yield from 2.2 mg/L refolding volume to 5.69 mg/L refolding volume. This suggests that high local concentration of TOL2 during the elution from the AEX column might have a negative effect on the protein in its monomeric form.

### Size Exclusion Chromatography (SEC)

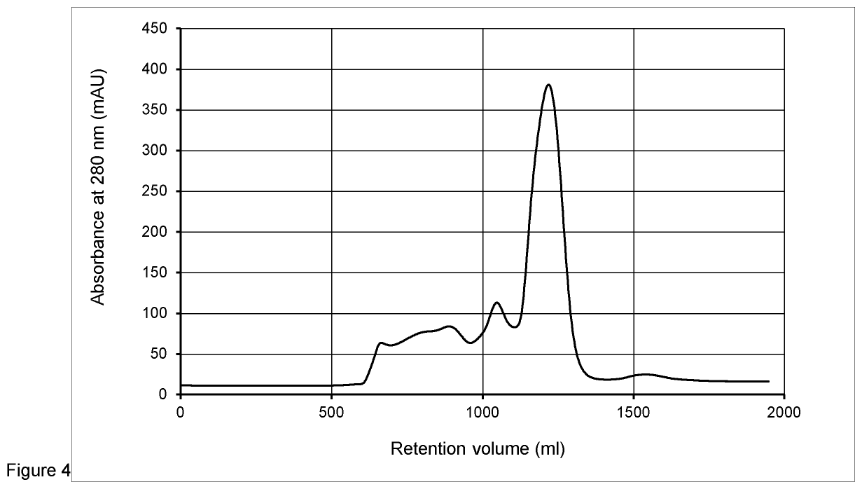

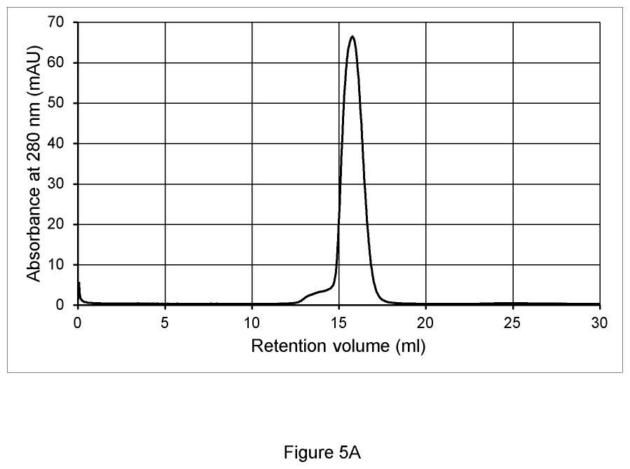

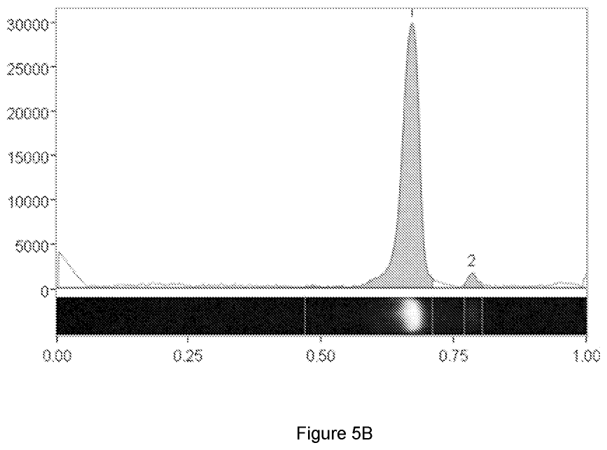

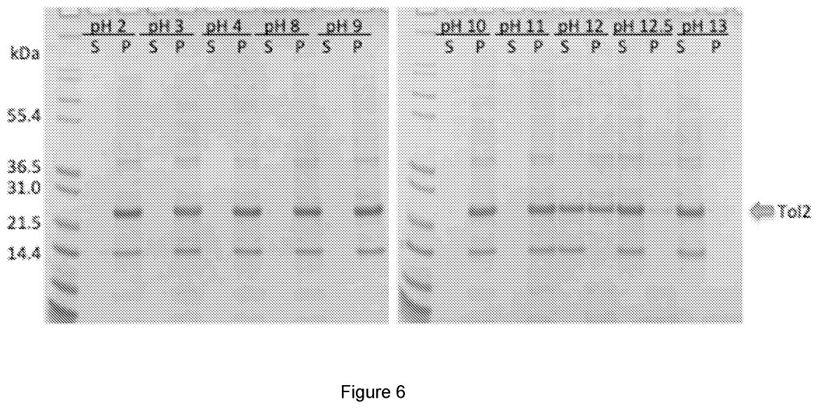

Eluted TOL2 was concentrated using a Biomax 10 kDa Pellicon XL cassette before loading onto a Superdex200 μg XK 50/890 equilibrated with 40 mM Tris, 150 mM NaCl, pH 8.5. TOL2 eluted primarily as a monomer at 1200 ml, but also dimeric and multimeric forms existed (FIG. 4). Monomeric TOL2 was concentrated to ca 2.5 mg/ml, sterile filtered and stored at −80° C. in aliquots. Analysis of the sample showed >95% was monomeric TOL2 (FIG. 5).

### Example 3

In this Example, the effect of pH on solubilization of TOL2 inclusion bodies was observed. 2.4 mg inclusion bodies were re-suspended in 1 mL PH X, 300 mM NaCl solution, where X is a pH between 2 and 13 in steps. The subsequent solution was centrifuged and both the supernatant (S) and pellets (P) were analyzed by observation of TOL2 protein on SDS-PAGE gels (FIG. 5). It was observed that TOL2 inclusion bodies started to be solubilized at pH 12 with full solubilization after pH 12.5.

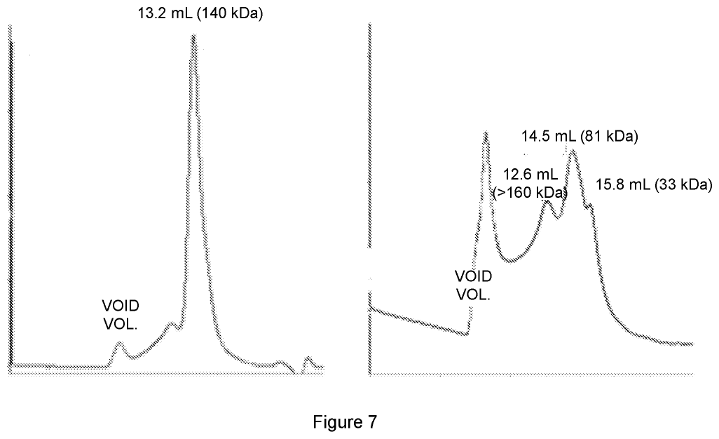

Analytical size exclusion on Superdex 200 HR 10/30 on inclusion bodies solubilized either with 50 mM Tris-HCl, 300 mM NaCl, 5 mM EDTA, 7 M urea, pH 8.0 (left in FIG. 7) or 0.0316 M NaOH, 300 mM NaCl, pH 12.5 (right in FIG. 7) indicated differences in the solubilization at different pHs.

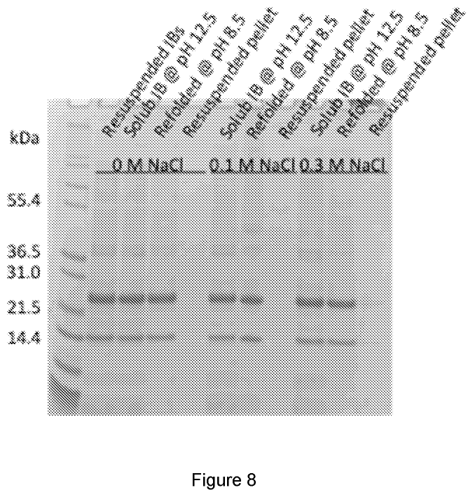

1 mL TOL2 solubilized at pH 12.5 was refolded at pH 8.5. Analysis on a non-reduced SDS-PAGE indicated that refolded TOL2 was soluble, that it refolded with and without NaCl and that refolded TOL2 did not have any intermolecular disulfides (FIG. 8).

The results from this Example were used to select optimal solubilization and refolding buffers as used in Example 1.

### Example 4

In this Example, conditions for anion-exchange chromatography of TOL2 were investigated.

Columns were loaded with 10 mg of total TOL2 protein per ml of Workbeads 40Q resin from solubilized inclusion bodies containing TOL2. The protein concentration was at 1-2 mg total protein/ml (measured by A280). All runs had the same occupancy time (5 min) to match the conditions used at scale. The column volume was 5 ml and the fraction volume was 1 ml (BBQ=BabyBioQ prepacked with Workbeads 40Q column from BioWorks). Loading experiments with and without 100 mM cysteine as reducing agent in the solubilization and running buffers were performed.

Loading Experiment with 100 mM Cysteine
Solubilization buffer: 10 mM glycine, 8 M urea, 5 mM EDTA, 100 mM cysteine, pH 9.5
Sample concentration: 1.408 mg/ml
Occupation time on column: 5 min Sample volume: 35 ml (50 mg total protein, 10 mg/ml resin)

The column volume was 5 ml and fraction volume 1 ml. Selected fractions were analyzed on reduced SDS-PAGE.

TOL2 inclusion bodies (1.79 g) were re-suspended in 120 ml solubilization buffer (10 mM glycine, 8 M urea, 5 mM EDTA, 100 mM cysteine, pH 9.5). A handheld mixer (ULTRA-TURRAX®) was used to dissolve inclusion body clumps. The mixture was stirred at room temperature for 2 h and sonicated 4×2 min total time (15 s on, 5 s off, 70% amplitude) while kept on ice to re-suspend the inclusion bodies completely. Unsolubilized material was pelleted by centrifugation (38,000 g, 1.5 hm 4° C.). 1.408 mg/ml solubilized TOL2 was obtained at a total volume of 112 ml.

The sample was loaded via peristaltic pump connected to the ÄKTA to observe the absorbance over time while not contaminating the FPLC system.
Sample: 1.408 mg/ml, 35.5 ml, 50 mg
Column BW_BBQ, 5 ml
Residence time: 5 min
Flow rate: 1 ml/min
Fraction size: 1 ml
Loading experiment without 100 mM cysteine
Solubilization buffer: 10 mM glycine, 8 M urea, 5 mM EDTA, pH 9.5
Sample concentration: 1.694 mg/ml
Occupation time on column: 5 min
Sample volume: 30 ml (50 mg total protein, 10 mg/ml resin)

The column volume was 5 ml and fraction volume 1 ml. Selected fractions were analyzed on reduced SDS-PAGE.

TOL2 inclusion bodies (1.18 g) were re-suspended in 100 ml solubilization buffer (10 mM glycine, 8 M urea, 5 mM EDTA, pH 9.5). A handheld mixer (ULTRA-TURRAX®) was used to dissolve inclusion body clumps. The mixture was stirred at room temperature for 2 h and sonicated 4×2 min total time (15 s on, 5 s off, 70% amplitude) while kept on ice to re-suspend the inclusion bodies completely. Unsolubilized material was pelleted by centrifugation (38,000 g, 1.5 hm 4° C.). 1.694 mg/ml solubilized TOL2 was obtained at a total volume of 112 ml.

The sample was loaded via peristaltic pump connected to the ÄKTA to observe the absorbance over time while not contaminating the FPLC system.
Sample: 1.694 mg/ml, 30 ml, 50 mg
Column BW_BBQ, 5 ml
Residence time: 5 min
Flow rate: 1 ml/min
Fraction size: 1 ml

### Results

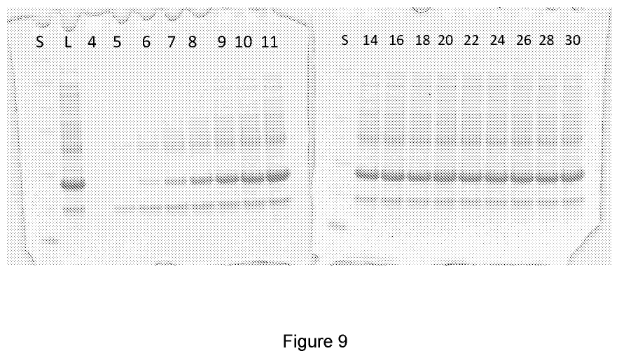

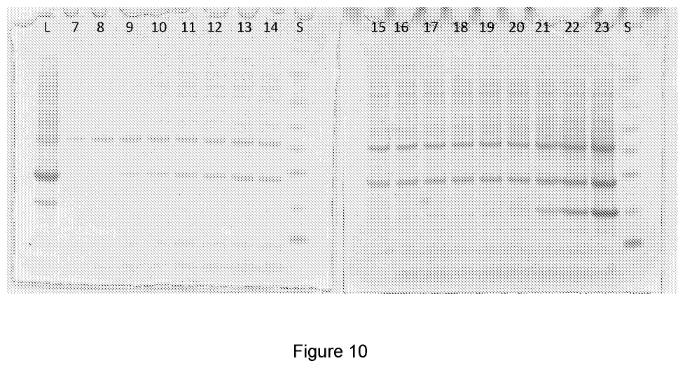

The column overload was delayed in the absence of 100 mM cysteine (FIGS. 9 and 10). In the experiment with 100 mM cysteine, TOL2 overloading started around 1.5 mg/ml resin and reached a maximum around 2.5 mg/ml (FIG. 9), whereas TOL2 overloading started around 3.75 mg/ml resin and reached a maximum around 7.5 mg/ml in the experiment without 100 mM cysteine (FIG. 10).

This Example showed that the TOL2 binding capacity of the resin and thereby yield of the TOL2 protein was increased by omitting the reducing agent (cysteine). This was highly surprising since TOL2 contains four cysteines and it is generally believed that ion-exchange chromatography of cysteine-containing proteins should be performed in the presence of a reducing agent to prevent formation of undesired intra-disulfide bonds between cysteine residues in the same protein molecule and/or inter-disulfide bridges between cysteine residues in different protein molecules.

### Example 5

This Example investigated anion-exchange chromatography purification of TOL2.

### Solubilization

˜10 g pellet of TOL2 inclusion bodies was solubilized in ca. 300 ml 10 mM glycine, 8 M urea, 5 mM EDTA, pH 9.5. The pellet was re-suspended using a homogenizer at room temperature (10 cycles of 10 seconds) and incubated for 2 h with magnetic stirring. Unsolubilized material was pelleted by centrifugation (38,000 g, 1.5 h, 4° C.). The TOL2 protein concentration after solubilization was 2.9 mg/ml (Pierce 660 nm protein assay), in a total volume of 300 ml, given about 0.9 g TOL2.

### Anion-Exchange Chromatography (AEX)

AEX buffers were prepared. 260 ml Workbeads 40Q column was washed with 2 M NaCl and Milli-Q water.
Resin: Workbeads 40Q, Column: 260 ml XK 26/40
Residence time: >5 min, Flowrate: ˜ 15 ml/min
Equilibration: Using the above solubilization buffer (on ÄKTA)
Volume: 2 column volumes (CV) (until the UV & conductivity are constant)
Load: 4 g/L resin i.e., 4 mg/ml=1.04 g of protein needs to be loaded onto the 260 ml column. However, ˜0.9 gm protein was loaded onto the AEX column.
System: Done using the concentration pump (highest flow rate)
Wash 1: Solubilization buffer+100 mM cysteine—2 CV
AEX buffer A: 10 mM glycine, 8 M urea, 5 mM EDTA and 20 mM cysteine, pH 9.5
AEX buffer B: 10 mM glycine, 8 M urea, 5 mM EDTA, 20 mM cysteine and 1 M NaCl, pH 9.5
Wash 2: Step gradient on ÄKTA: 100% AEX buffer A—0% of AEX buffer B—3 CV (until the UV & conductivity stabilizes)
Wash 3: Step gradient on ÄKTA: 93% AEX buffer A—7% of AEX buffer B—3 CV
Elution: 75% AEX buffer A—25% AEX buffer B—3 CV
Strip: 100% AEX buffer B—2 CV

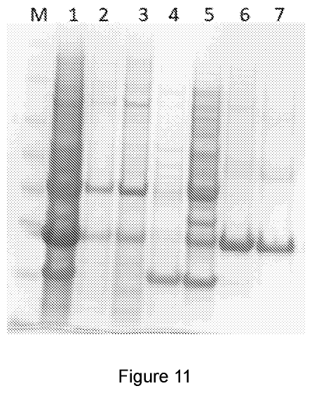

This Examples showed that a TOL2 loading of around 4 mg protein per ml of Workbeads 40Q resin did not overload the column, see lane 2 in FIG. 11.

### Example 6—Production and Purification of Monomeric TOL2

This example describes a process of producing and purifying monomeric TOL2.

### Solubilization

TOL2 inclusion bodies were re-suspended in solubilization buffer (10 mM glycine, 8 M urea, 5 mM EDTA, pH 9.5) at 1 g TOL2 inclusion body per 30 ml solubilization buffer. The solution was homogenized for 10 cycles of 10 s and stirred for 2 hours.

### Anion-Exchange Chromatography 1 (AEX1)

Anion-exchange chromatography was performed for the solubilized TOL2.
Resin: Workbeads 40Q, Column: XK 50/30
Residence time: 4.8 min, Flowrate: 130.9 ml/min
Equilibration: 5 CV, solubilization buffer (10 mM glycine, 8 M urea, 5 mM EDTA, pH 9.5)
Load: 4 g TOL2/L resin
Wash 1: 5 CV, AEX-1 buffer A, no cysteine (10 mM glycine, 8 M urea, 5 mM EDTA, PH 9.5)
Wash 2: 5 CV, AEX-1 buffer A (10 mM glycine, 8 M urea, 5 mM EDTA, 10 mM cysteine, pH 9.5)
Wash 3: 5 CV, 93% AEX-1 buffer A, 7% AEX-1 buffer B (10 mM glycine, 8 M urea, 5 mM EDTA, 10 mM cysteine, 1 M NaCl, pH 9.5)
Elution: 10 CV, 75% AEX-1 buffer A, 25% AEX-1 buffer B
Strip: 6 CV, AEX buffer B

### pH Adjustment

1 M NaOH was added dropwise to the TOL2 eluate from AEX1 to a final pH of 11.5 under stirring at room temperature.

### Refolding

pH-adjusted TOL2 eluate was added dropwise into a refolding buffer (0.1 M Tris-HCl, 5 mM EDTA, 5 mM cysteine, 0.4 M sucrose, 10% glycerol, pH 8.6) to achieve a dilution ratio of 1/5 (1 volume of pH-adjusted TOL2 eluate+4 volumes of refolding buffer) for a total addition time of 2 hours, at a temperature <10° C. The refolding TOL2 was incubated 6-10 hours with magnetic stirring, at a temperature <10° C.

### Filtration

The refolded TOL2 was filtrated through a 0.2 μm Sartopore 2 Midicap filter at a temperature <10° C. and the filtrate was collected.

### Anion-Exchange Chromatography 2 (AEX2)

Anion-exchange chromatography was performed for the refolded TOL2.
Resin: Q Sepharose FF, Column: VL22/250
Residence time: 4.8 min, Flowrate: 11.9 ml/min
Equilibration: 5 CV, equilibration buffer (20 mM Tris-HCl, 35 mM NaCl, 0.01% Pluronic, pH 8.5)
Load: 10 g TOL2/L resin
Wash 1: 5 CV, AEX-2 buffer A (20 mM Tris-HCl, 35 mM NaCl, 0.01% Pluronic, pH 8.5)
Wash 2: 5 CV, 85% AEX-2 buffer A, 15% AEX-2 buffer B (20 mM Tris-HCl, 1 M NaCl, 0.01% Pluronic, pH 8.5)
Elution: 10 CV, 70% AEX-2 buffer A, 30% AEX-2 buffer B
Strip: 5 CV, 40% AEX-2 buffer A, 60% AEX-2 buffer B

### Tangential Flow Filtration (TFF)

Equipment: Sartorius Sartoflow Smart
Cassette: Pellicon XL Biomax
Membrane material: PES
Cut-off: 10 kDa
Area: 0.005 m²

The system was equilibrated with 10 L/m² water for injection (WFI) and 10 L/m² buffer (40 mM Tris-HCl, 150 mM NaCl, pH 8.5). The TOL2 eluate from AEX2 was concentrated to approximatively 2.5-3.0 g/L.

### Filtration

The concentrated TOL2 was filtrated through a 0.2 μm Sartopore 2 Midicap filter and the filtrate was collected.

### Size Exclusion Chromatography (SEC)

Resin: Superdex 200 μg, Column: XK 26/1000
Residence time: 129.4 min, Flowrate: 3.2 ml/min
Equilibration: 35 CV, equilibration buffer (40 mM Tris-HCl, 150 mM NaCl, pH 8.5)
Load: 2-4% CV
Elution: 2 CV SEC buffer (40 mM Tris-HCl, 150 mM NaCl, pH 8.5)

### Filtration

The SEC eluate was filtrated through a 0.2 μm Sartopore 2 Midicap filter and the filtrate was collected.

### Example 7

Myasthenia gravis (MG) is a CD4+ T cell-dependent antibody-mediated autoimmune disease, which leads to destruction of the skeletal muscle nicotinic acetylcholine receptor (AChR) at the neuromuscular junction resulting in the hallmark MG symptoms of muscle weakness and fatigue. Antibodies against the AChR are found in a majority of patients (˜85%), while fewer patients have antibodies against the muscle specific kinase (˜9%), the low-density lipoprotein receptor-related protein 4 (˜2%), or other less common targets. The AChR is a Q14 transmembrane glycoprotein composed of five subunits with a stoichiometry of (α1)2B1γδ in fetal or denervated muscles and (α1)2B1εδ in adult muscles. Each subunit has a highly structured extracellular domain (ECD), which contains the disease-relevant autoantibody binding sites. Among the different ECDs, that of the α1 subunit (α1-ECD) is the primary antibody target in the autoimmune attack and most evidence so far suggests that the α1-ECD directed antibodies are the most pathogenic. Although MG is an antibody-mediated disease, high affinity autoantibody production by B cells is dependent on CD4+ T cell activity. Indeed, AChR-reactive CD4+ T cells have been found in MG patients, while T cell recognition of the AChR has been examined extensively and several studies have identified T cell reactive auto-peptides, in particular from the α1-ECD. Taken together, both the antibody and T cell reactivities point to the α1-ECD as being a disease-specific antigen of particular interest in MG.

Currently, the most common therapeutic strategies for MG include the use of cholinesterase inhibitors, corticosteroids, immunosuppressants, plasmapheresis, intravenous immunoglobulin, monoclonal antibodies or thymectomy. These treatments are not disease-specific and can cause significant side-effects. They can alleviate symptoms, but they are not curative. Therefore, lifelong immunosuppressive therapy is often required but some patients may prove treatment refractory.

The ideal therapy would be disease-specific and target efficiently only the pathogenic autoreactive component of the immune system. Antigen-specific immune tolerization for treatment of autoimmune diseases may specifically abrogate autoimmunity without hampering normal immune function.

Experimental autoimmune MG (EAMG), which can be induced in rats by administration of AChR domains, represents a reliable animal model for the study of novel therapeutics against MG. Early attempts to induce antigen-specific immune tolerance in EAMG rats involved oral or nasal administration of AChR subunit domains. Both preventive, i.e., treatment before disease induction, and therapeutic treatment regimens have been explored, with the latter requiring higher antigen doses than the former to achieve a comparable effect. Mucosal administration of AChR-derived peptides, rather than whole protein domains, has also been tested with some positive results in EAMG mice.

Induction of tolerance via intravenous injection (i.v. tolerance) is also possible. Notably, administration of soluble or nanoparticle-carried antigens via the intravenous route has recently rendered positive results against autoimmune diseases, such as multiple sclerosis, Graves' disease, and celiac disease, in the clinic, pointing to the intravenous route of administration as promoting a tolerogenic setting suitable for antigen-specific immune tolerance approaches.

In this Example, we sought to develop a soluble protein-based intravenous therapeutic approach for MG, suitable for translation to the clinic, combining the potential advantages of allowing patients' own antigen processing and presentation.

To this end, we used a rat EAMG model induced with recombinant domains of the human AChR, previously used for the study of antigen-specific treatments, and explored the therapeutic efficiency of intravenous antigen administration, which has not been studied in MG before. We found that intravenous administration had a robust therapeutic effect in EAMG, contrary to current MG therapeutics. We proceeded to characterize in detail several parameters affecting treatment efficacy, such as dose of antigen and frequency of administration. We conclude that this approach could be developed as a novel highly effective treatment for MG.

### Materials and Methods

### Experimental Animals

6- to 7-week-old female Lewis rats (weighing 120-135 g) were obtained from the animal breeding unit of the Department of Animal Models for Biomedical Research of the Hellenic Pasteur Institute. They were maintained in the rodent unit of the Department, in plastic cages with wire mesh lids and 4 cm thick wood-shavings bedding (four rats per 1,600 cm² cage). Upon symptom manifestation they were provided with water gels and soft food at the bottom of the cages throughout the remaining experiment. All experiments described were approved by the Institute Ethics Board and conducted according to the regulations and guidelines for animal care (EU Directive 2010/63/EU for animal experiments).

Synthesis of α1-ECDₘₜ and α1-ECDₘ

α1-ECDₘₜ consists of the ECD of the human AChR α1 subunit, mutated by having its Cys-loop exchanged for that of the homologous acetylcholine binding protein from the snail Lymnaea stagnalis, and tagged with a Flag-tag and a 6-His-tag at its N- and C-terminal ends, respectively. α1-ECDₘₜ was expressed in the yeast Pichia pastoris as a soluble secreted polypeptide and purified by means of metal-affinity chromatography followed by size exclusion chromatography as previously described (Int J Biol Macromol (2014) 63: 210-217). Except for the Flag- and His-tags, the amino acid sequence of α1-ECDₘ (TOL2, SEQ ID NO: 6) consists of the same elements as α1-ECDₘₜ. α1-ECDₘ was expressed in Escherichia coli strain NEB express (New England Biolabs Inc. USA) using a modified version of the pTrc99A-vector (Pharmacia AB, Sweden) harboring the km^{r} gene and in which the gene encoding α1-ECDₘ was under transcriptional control by an IPTG-inducible Trp/Lac promoter. Briefly, E. coli cells were cultured in terrific broth (Thermofisher Scientific, USA) at 37° C. and α1-ECDₘ expression was induced with 1 mM IPTG at an OD₆₀₀ of about 0.6. Following induction, α1-ECDₘ accumulated in inclusion bodies in high quantities. α1-ECDₘ-containing inclusion bodies were purified by cell-disruption in lysis buffer (0.1 M Tris, 5 mM EDTA, pH 8.5) followed by repeated washings in 2 M Urea, 2% Triton X-100 in lysis buffer and finally solubilized in 40 mM Tris, 8 M Urea, 5 mM EDTA, pH 8.5. Refolding of α1-ECDₘ was performed in 40 mM Tris, 50 mM NaCl, 1 M Urea, 10% Glycerol, 5% Sucrose pH 8.5 overnight at 4° C. Refolded α1-ECDₘ was further purified by anion exchange chromatography on Q Sepharose FF at pH 7.4 and size exclusion chromatography (SEC) on Superdex 200 μg. Following the SEC step, α1-ECDmpurity was >90% with endotoxin levels below <1 EU/mg. The overall yield of purified α1-ECDₘ was about 80 mg/L of E. coli culture. Following sterile filtration, α1-ECDₘ was frozen in storage buffer (30 mM NaP, 0.3 M NaCl, pH 7.4) and stored at −80° C.

### Induction, Treatment, and Clinical Evaluation of EAMG

For induction of EAMG, rats were anaesthetized with 2% isoflurane supplemented with oxygen. They were injected subcutaneously in both hind footpads and at three sites in the lower back with a total of 80 mg α1-ECDₘₜ prepared as described above, or PBS for controls, in CFA (Becton, Dickinson and Company) supplemented with 2 mg/ml inactivated Mycobacterium tuberculosis H37RA (Becton, Dickinson and Company), in a final volume of 250 ml. Serum samples were collected from the rats by tail vein blood sampling at different timepoints during the experiments and used for anti-AChR antibody quantitation as described in Statistical Analysis.

Regarding treatment administration, rats were treated intranasally or intravenously with α1-ECDₘₜ or α1-ECDₘ starting 7, 21 or 40 days after EAMG induction. The amount of protein was 100 μg administered in a volume of 10 μl per nostril, or 100, 500 or 1,000 μg administered in a volume of 200 μl in tail vein. For the experiments comparing α1-ECDₘₜ with MG current standard of care, rats were treated with 1 mg methylprednisolone (Solumedrol, Pfizer) injected IP in a volume of 100 ml or 18.5 mg/kg pyridostigmine (Mestinon, Meda Pharma GmbH), administered via oral gavage in a volume of 200 ml. These doses were higher than what is commonly used in clinical practice, but well tolerated in EAMG. A list of all the applied regimens is shown in Table 3. Control animals received only PBS in all experiments.

TABLE 3
Overview of the different treatment regimens applied

| | | | Amount per dose |
|---|---|---|---|
| | | | (doses distributed |
| | Administration | Start of | over 12 days |
| Drug/Antigen | route | administration | in all cases) |
| α1-ECDₘₜ | intranasal | D7 | 100 μg (12 doses) |
| | intravenous | D7 | 5 μg, |
| | | | 25 μg or 100 μg |
| | | | (12 doses) |
| | | | 200 μg (6 doses) |
| | | | 100 μg (6 doses) |
| | | | 400 μg (3 doses) |
| | | | 200 μg (3 doses) |
| | intravenous | D21 | 100 μg or 500 μg |
| | | | (12 doses) |
| | intravenous | D40 | 500 μg or 1000 μg |
| | | | (12 doses) |
| α1-ECDₘ | intravenous | D21 | 100 μg or 500 μg |
| | | | (12 doses) |
| | intravenous | D40 | 500 μg or 1000 μg |
| | | | (12 doses) |
| pyridostigmine | Intragastrical | D40 | 18.5 mg/kg |
| | | | (12 doses) |
| methylprednisolone | intraperitoneal | D40 | 1 mg (12 doses) |

The rats were monitored once a week for the first 4 weeks after EAMG induction and daily thereafter. Body weight was recorded and clinical score was observed on a flat bench before and after exercise and graded based on the presence of the following symptoms: tremor, hunched posture, reduced strength/mobility and dropped head. Exercise consisted of repetitive grasping and pulling of a 350 g grid while being held by the base of the tail for 30 seconds (Muscle Nerve (1990) 13: 485-492). EAMG scores were evaluated as follows: 0: normal strength, no symptoms; 1: normal before exercise, symptoms observed after exercise due to fatigue; 2: symptoms present without exercise; 3: severe symptoms at rest, hind limb paralysis, no grip; 4: moribund (Exp Neurol (2015) 270:18-28). To minimize investigator bias, the animals were scored by two investigators, one of which was blinded to the treatment groups, and the average scores were used in the analyses.

### Electromyography

The rats were anaesthetized with 2% isoflurane. To measure the compound muscle action potential (CMAP) the tibialis anterior muscle was examined. A grounding electrode was placed subcutaneously at the upper back; a stimulating electrode was inserted at the base of the tail to stimulate the sciatic nerve; a recording electrode was placed in the center of the tibialis muscle and a reference electrode more distally at the tendon. A set of 10 supramaximal stimuli at 3 Hz were delivered and the CMAP recorded. The average decrement for each muscle was calculated from at least three separate readings.

### α1-ECDₘₜ Distribution Studies

¹²⁵I-labeled α1-ECDₘₜ equivalent to 106 cpm was mixed with unlabeled α1-ECDₘₜ to a total of 100 μg protein, which was injected intravenously to healthy or EAMG rats. Blood samples were collected from the tail artery at specific time points and organs were collected for analysis 6 hours after injection. Radioactivity was measured in a 1470 Wizard g-counter. To calculate the organ distribution, the labelling attributed to the blood content of each organ was estimated and subtracted (Mol Pharmaceut (2014) 11:1591-1598).

### RIPA for Rat AChR and α1-ECDₘₜ Antibody Quantitation

α1-ECDₘₜ or a-bungarotoxin (Sigma-Aldrich, USA) were labeled with ¹²⁵I using the chloramine T method. Following, the antibodies in test serum samples were quantified using RIPA. In brief, for the detection of α1-ECDₘₜ antibodies, ¹²⁵I-α1-ECDₘₜ (50,000 cpm) was incubated for 2 h at 4° C. with serial dilutions of the test serum (made in normal rat serum). The total volume of rat serum used was 2 ml. Then 10 ml of rabbit anti-rat serum were added and incubated overnight at 4° C. Finally, the samples were washed twice with PBS-T and the remaining radioactivity measured in a 1470 Wizard g-counter. The dilutions showing a linear increase were used for the calculation of the antibody titers. For the detection of rat AChR antibodies, rat AChR was prepared from denervated rat muscle and labeled for 1 h at 4.C with ¹²⁵I-cx-bungarotoxin (50,000 cpm) before incubation with the test serum serial dilutions. All the following steps were performed as described previously for the α1-ECDₘₜ antibodies.

To calculate the antibody titers in nM, the following formula was used:

$$Antibody titer = ( cpm test serum sample - cpm of normal rat serum ) / ( specific activity of labeled antigen \times serum volume in μl \times dilution factor ) ,$$

$$where specific activity = cpm / fmol labeled antigen :$$

### Statistical Analysis

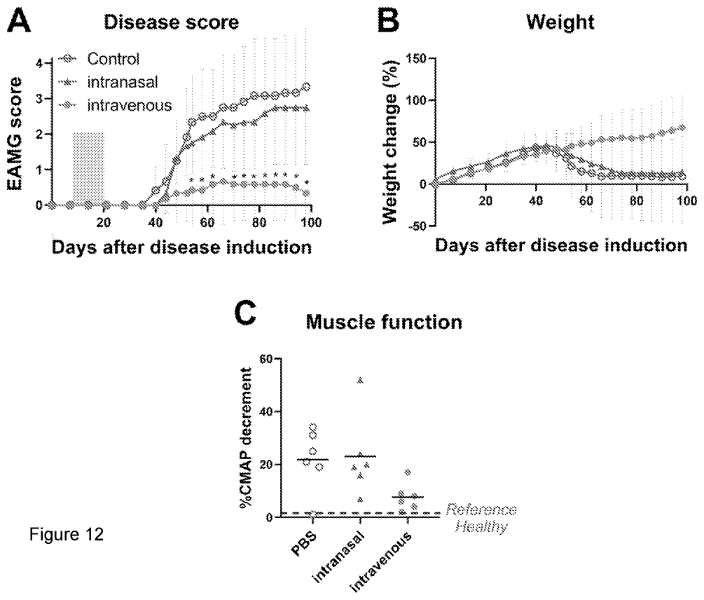

Statistical analysis was performed using GraphPad Prism (GraphPad Software, La Jolla, CA). In FIGS. 12-16 when comparing at least three groups, for EAMG scores and CMAP decrement the two-tailed nonparametric Kruskal Wallis test was used. All other comparisons were made using two-tailed ANOVA.

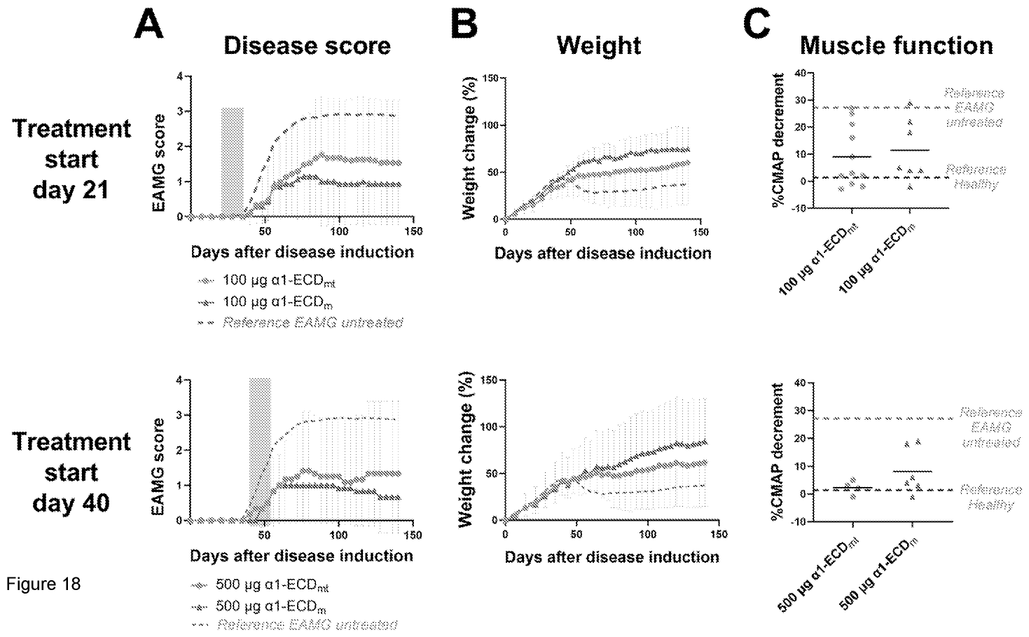

In FIG. 18 when comparing only two groups, for EAMG scores and CMAP decrement the two-tailed nonparametric Mann Whitney test was used. All other comparisons were made using unpaired t-test. For all figures: ****p<0.0001, ***p<0.001, **p<0.01, *p<0.05.

### Results

### Administration of α1-ECDₘₜ Via the Intravenous Route Greatly Enhances Treatment Efficacy

We used a mutated and tagged version of the α1-ECD with vastly improved solubility (α1-ECDₘₜ), near native conformation and practically identical binding to autoantibodies from MG sera, compared to the wt protein (Int J Biol Macromol (2014) 63: 210-217, J Neuroimmunol (2015) 278:19-25) that could be used as an antigen-specific tolerogen. Initially, we investigated the potential of therapeutic intravenous antigen administration, using our robust rat model of EAMG.

To this end, we treated EAMG rats daily on 12 consecutive days from day 7 after disease induction with 100 mg of α1-ECDₘₜ, administered by intravenous injection or intranasal droplets. We observed a highly significant improvement in treatment effect after intravenous administration compared to intranasal administration (FIG. 12). In fact, most of the intravenously α1-ECDₘₜ-treated rats remained asymptomatic (4/6 had an EAMG score of 0), while 5/6 of the intravenously PBS-treated rats presented with very severe disease. Similarly, the body weight and CMAP measurements of the intravenously treated animals were improved (FIGS. 12B, 12C).

### α1-ECDₘₜ Abrogates EAMG Development in a Dose and Time Dependent Manner

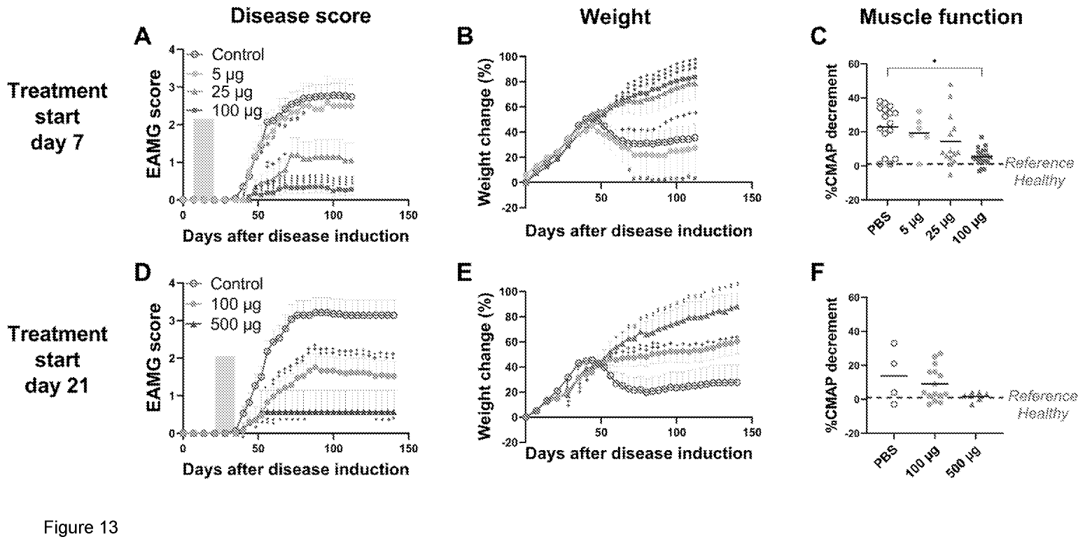

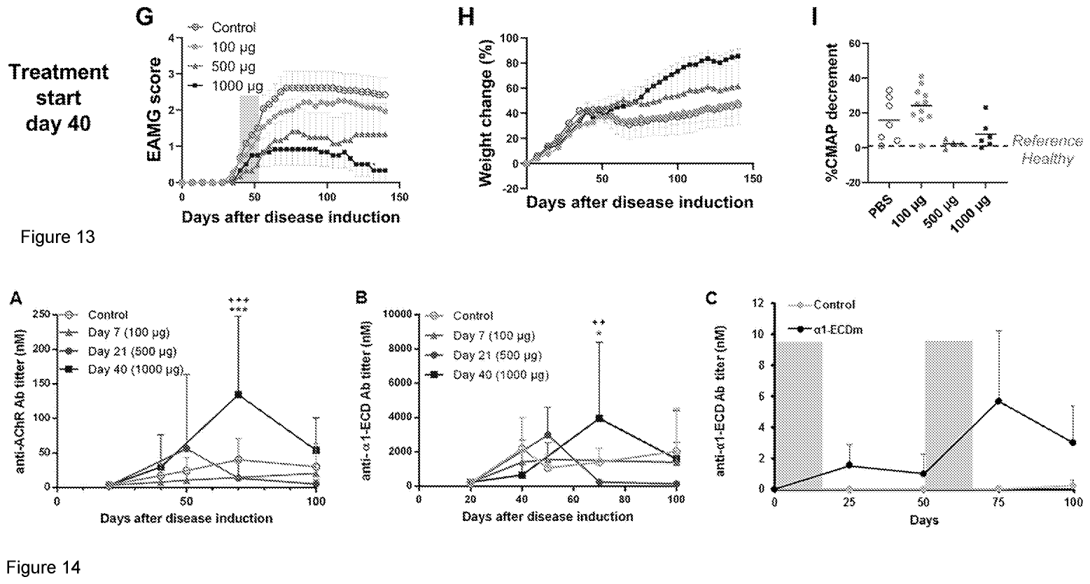

To investigate if α1-ECDₘₜ demonstrated a dose-response relationship we injected the drug candidate at different doses ranging from 5 μg to 100 μg, starting treatment on day 7 as previously. We observed a strong correlation between the dose of α1-ECDₘₜ and treatment outcome, with higher doses having increased therapeutic efficacy. Indeed, rats treated with the 5 mg dose showed minimal improvement as their EAMG score reached 2.58 (±0.72), while those that received 25 mg and 100 mg had an average maximum score of 1.2 (±0.45) and 0.35 (±0.16) respectively (FIG. 13A). These findings were corroborated by the changes seen in body weight and CMAP decrement of the rats (FIGS. 13B, 13C).

Since the ultimate goal of antigen-specific tolerization is to treat ongoing autoimmune disease, we further studied the therapeutic effect of treatment with α1-ECDₘₜ following intravenous administration initiated at later timepoints after EAMG induction, specifically day 21 or 40. At these timepoints, EAMG rats display progressive disease both at the molecular and the clinical level, at different severities (subclinical to first symptom appearance). We found that treatment starting at these later time points was still effective but required higher drug doses to achieve comparable treatment effects to those obtained at earlier time points. As seen in FIG. 13D, administration of 100 mg doses per day starting on day 21, resulted in an improvement of the EAMG score compared to mock-treated rats, but less than when treatment was initiated on day 7. However, increasing the daily dosing to 500 mg α1-ECDₘₜ initiated at day 21, had a more robust effect. Similarly, when treatment was initiated on day 40, the 100 mg dose group was less effective and not different from the PBS controls, while at higher doses of 500 mg or 1000 mg a profound improvement was achieved (FIG. 13G). As previously, further evidence to these findings was given by measurements of the changes in body weight and CMAP decrement (FIGS. 13E, 13F, 13H, 13I). Following, we measured the rat AChR and α1-ECDₘₜ antibody titers of the highest dosed animals at different time points. We saw that treatment resulted in reduction of the AChR antibodies compared to PBS treated rats when administered on day 7, while treatment on days 21 or 40 post induction resulted in an antibody increase (FIG. 14A). Similarly, α1-ECDₘₜ antibodies showed an increase in rats treated at the later time points (FIG. 14B). However, there was no correlation between disease score and levels of total rat AChR antibodies or α1-ECDₘₜ antibodies upon treatment, suggesting specific antibody characteristics influence their role. Furthermore, healthy 6 week-old rats injected with 500 mg protein i.v. in two sessions (12 daily injections each session) developed low levels of α1-ECDₘₜ antibodies, did not have detectable rat AChR antibodies and did not develop EAMG symptoms (FIG. 14C). Notably, at the highest dose investigated, with a short-term dosing regimen, applied at a time point of established disease, α1-ECDₘₜ could ameliorate disease symptoms.

### EAMG Treatment Efficacy of α1-ECDₘₜ Depends on Dosing Frequency

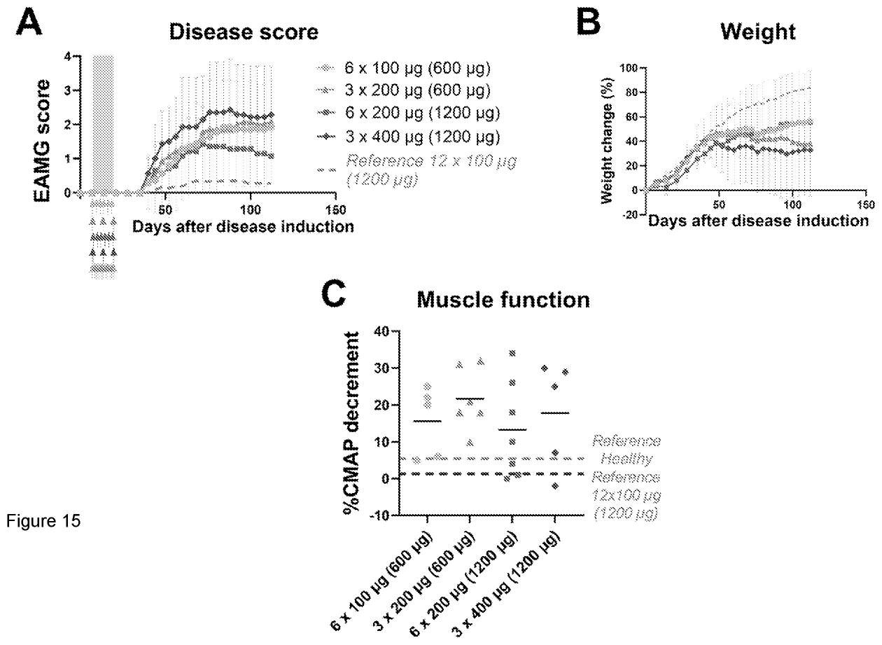

We next sought to determine the effect of dosing schedule and the impact on treatment efficacy in relation to dosing intervals. To this end, we compared the therapeutic effect of α1-ECDₘₜ injected intravenously in EAMG rats, every other day or every four days, over a twelve-day-period. The total amount of α1-ECDₘₜ administered over the entire treatment period was either 1200 mg (in the 6×200 mg and 3×400 mg groups) or 600 mg (in the 6×100 mg and 3×200 mg groups). The results obtained indicate that treatment effect of frequent administrations of lower doses of α1-ECDₘₜ is superior to less frequent administrations of higher doses (FIG. 15). Indeed, 6 administrations of 200 mg was more effective compared to 3 administrations of 400 mg. Similarly, administration of 6×100 mg was somewhat better than 3×200 mg, or even 3×400 mg, although the difference was not as profound, possibly due to the lower total antigen dose administered (600 mg), which yielded an overall moderate therapeutic effect. Taken together, the therapeutic efficacy of ox-ECDₘₜ in the EAMG model is enhanced by continuous daily dosing of drug over 12 days compared to fewer administrations, of higher drug doses, over the same period.

EAMG Treatment Efficacy of α1-ECDₘₜ is Superior to that of Two Different Active Controls

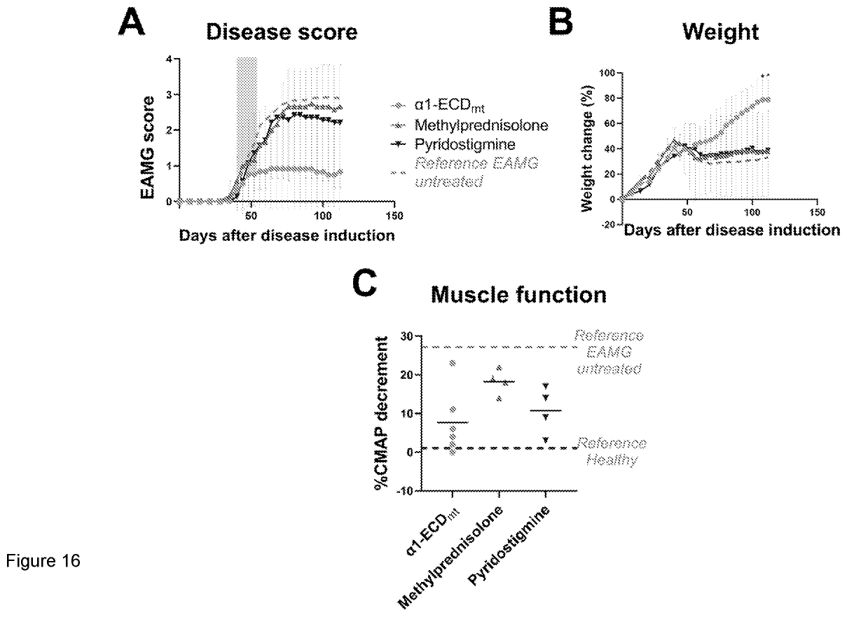

To evaluate whether α1-ECDₘₜ treatment could confer significant benefit in the EAMG model over current mainstay treatments for patients with MG, we compared it to a cholinesterase inhibitor (pyridostigmine) and a corticosteroid (methylprednisolone) commonly used in clinical practice. As seen in previous experiments, treatment with α1-ECDₘₜ starting on day 40 resulted in remission of EAMG symptoms (FIG. 16). In contrast, treatment with the cholinesterase inhibitor or the corticosteroid over the same period had reduced efficacy compared to the test antigen.

### α1-ECDₘₜ has a Short Plasma Half-Life and is Distributed to the Liver, Kidneys, and Spleen Upon Intravenous Administration

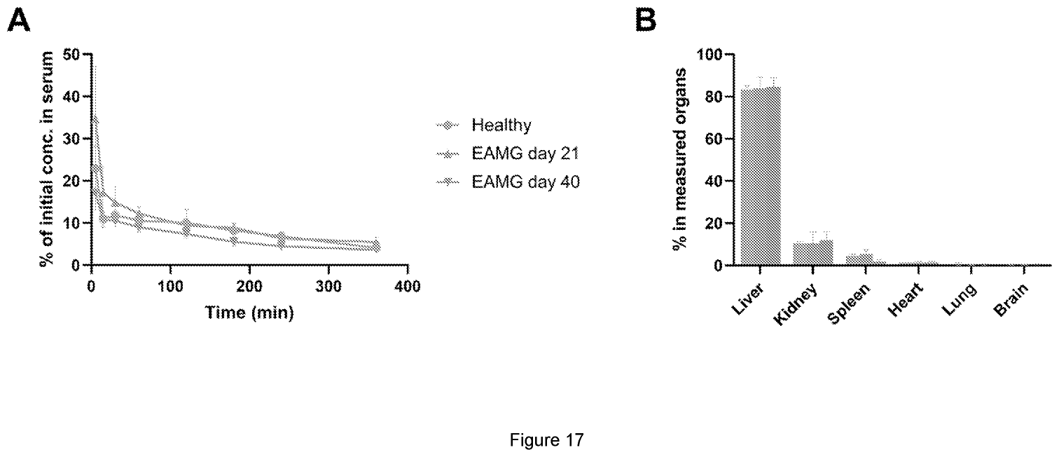

Upon intravenous administration of α1-ECDₘₜ to either healthy or EAMG rats, on day 21 or day 40 after disease induction, plasma levels of α1-ECDₘₜ followed a biphasic curve, with a steep distribution phase followed by a shallow elimination phase (FIG. 17A). Importantly, there was no significant difference in distribution and elimination of 1-ECDₘₜ after administration to either healthy or EAMG rats, indicating that the presence of α1-ECDₘₜ antibodies in the EAMG rats did not affect the drug pharmacokinetics.

We studied the organ distribution of α1-ECDₘₜ 360 min after intravenous injection into healthy or EAMG rats on day 21 or day 40 after disease induction. Uptake, calculated as the percentage of measured protein in all organs examined, was predominant in the liver, and to a lesser extent in the kidneys and spleen, while uptake into heart, lung, and brain was negligible (FIG. 17B). As for plasma clearance, organ distribution was not affected by the stage of disease development, nor by the absence or presence of α1-ECDₘₜ antibodies.

α1-ECDₘₜ and α1-ECDₘ Display Comparable Treatment Efficacies

In view of developing an antigen-specific therapy for MG suitable for use in the clinic, we re-engineered α1-ECDₘₜ by removal of the N- and C-terminal protein purification tags, and thus created α1-ECDₘ. These tags are extremely useful as research tools, enabling easy and efficient purification by affinity chromatography, but are inappropriate as parts of human protein therapeutics by posing an increased immunogenicity risk. Moreover, whereas α1-ECDₘₜ was produced in the yeast P. pastoris, we elected to produce α1-ECDₘ in E. coli for ease of manufacturing and scale-up purposes. In experiments comparing intravenous treatment of EAMG rats with α1-ECDₘ or α1-ECDₘₜ, starting on day 21 or day 40 after disease induction with 100 mg or 500 mg doses respectively, we found comparable treatment efficacies of the two proteins (FIG. 18). Taken together, these results obtained in the rat EAMG model point to α1-ECDₘ as an antigen-specific therapeutic for translation to the clinic as a promising drug candidate for the treatment of patients with MG.

### Discussion

Immunotherapies commonly used to treat MG are unspecific and associated with serious long-term side effects. To mitigate this, some immunotherapies currently in development are directed against pathological mechanisms more specific to MG. These therapies, such as complement C5 inhibitors and neonatal Fc receptor inhibitors, may be associated with fewer side effects, but they will not reinstate tolerance and are, thus, not long-lasting or curative. Antigen-specific immunotherapies aiming to restore tolerance to the autoantigen under attack by specifically targeting only the part of the immune system that has gone awry while leaving the rest intact are, therefore, considered the holy-grail for treatment of autoimmune diseases.

Antigen-specific tolerization approaches based on the α1-ECD have been studied earlier, using the oral or intranasal but not the intravenous route of administration. Delivery via the intravenous route could exploit a natural noninflammatory path, readily perfusing several organs with resident immune cells which have developed unique mechanisms for induction and maintenance of tolerance.

Comparison of intravenous and intranasal administration in our animal model showed that the former was much more potent in treating EAMG rats. The low efficacy of intranasal treatment observed compared to published data from other groups, could be due to the relatively high severity of disease induced in our model, which, if left untreated, results in >95% symptomatic animals and high average clinical scores. The effect of intravenous treatment was clearly dose dependent, similar to what is seen in studies on i.v. tolerance in the EAE animal model (Mult Scler (1999) 5(1): 2-9). This effect could be due to binding of the injected α1-ECDₘₜ to circulating antibodies. However, since previous studies on aphaeresis of autoantibodies have shown that the therapeutic effect only lasts a few days after treatment termination (J Neuroimmunol (2017) 312: 24-30), while in this case the benefit was long-lasting, we suggest that at least the lasting effect is mostly due to an additional mechanism. Although a high therapeutic effect at later time points during disease progression was more difficult to achieve, possibly due to accumulation of extensive damage at the neuromuscular junctions as well as establishment of memory cells, increased doses of administered antigen were capable of ameliorating disease symptoms. Importantly, administration of the antigen was always performed after disease induction, rather than before (preventive treatment) when antigen-specific cells and antibodies are not yet present.

To investigate the dose dependency and the requirement for repeated administrations, we examined the fate of α1-ECDₘₜ after intravenous administration. The pharmacokinetic properties of α1-ECDₘₜ with its very short plasma half-life could explain the need for larger doses and repeated administration as these measures may increase exposure of the protein to relevant tissues and cells involved. Pharmacokinetics of intravenously administered molecules is dictated by properties such as charge, size, shape, solubility and receptor interactions. Investigation of the protein's biodistribution in major organs after injection revealed that most of what remains in the body is found in key organs, known to be involved in tolerance, namely the liver, kidneys, and spleen.

We have conducted a preliminary non-GLP toxicological study, in which noninduced rats were dosed with 500 mg/rat/day of α1-ECDₘ or PBS (3 per treatment). Serum IFN-γ, TNF-α, IL-2, IL-6, IL-10 and CRP were measured the day after the last injection, and no differences in any of the cytokine or CRP levels were observed between the two animal groups (data not shown).

Peptides have been successfully used for induction of mucosal tolerance in mouse EAMG as well as for i.v. tolerance in rat EAMG in complexes with class II MHC molecules. A hurdle in designing antigen-specific drug candidates is the polyclonal complexity of autoimmune diseases driven by distinct, diverse autoreactive immune cell repertoires of patients. Indeed, studies on peptide-induced tolerance in EAMG showed that individual AChR peptides failed to produce any therapeutic benefit, despite tolerization against those specific AChR epitopes, highlighting the difficulty in tolerance spreading over bystander epitopes. The use of a protein autoantigen, presenting a majority of the autoepitopes involved in the autoimmune disease in their native context, allows for patients' individual antigen processing and presentation, thereby potentially reducing requirements associated with peptide-based approaches such as the need for dissection of immunodominant autoepitopes, production of personalized autoepitopes or patient stratification by HLA-type, and reliance on bystander effects to overcome patient heterogeneity.

Another reasoning for using T cell epitope peptides is to avoid administration of antigens bearing conformational epitopes that may be recognized by B cells. Studies in the rat EAMG model have shown that oral administration of an α1-ECD construct with more native conformation resulted in disease exacerbation, while treatment with a similar but less native fragment was able to suppress ongoing EAMG (J Immunol (Baltimore Md: 1950) (2000) 165: 3599-3605). However, in the case of intravenous administration, our results show that antigen conformation did not negatively affect the efficiency of treatment, since the P. pastoris protein used in most of the studies herein has a near native conformation, perhaps owing to differences in mechanism of action between the two administration routes.

While it is well understood that the molecular immunopathology in about 85% of patients with MG is due to the presence of circulating autoantibodies specifically targeting the AChR, the AChR antibody titer generally does not correlate with disease severity. Likewise, analysis of autoantibody levels in the rat EAMG model used here showed a weak correlation between disease score and the levels of total rat AChR antibodies measured in untreated animals and negligible correlation upon treatment. In both cases, in agreement with previous findings (J Neuroimmunol (2017) 303:13-21), there was negligible correlation between clinical score and total α1-ECD antibodies. The levels of total AChR and α1-ECDₘₜ antibodies were found increased in rats treated 21 or 40 days after disease induction. Similar results have been shown in some cases of oral administration, where improvement of clinical findings were accompanied by increased antibody titers (Ann New York Acad Sci (1996) 778: 258-272). These results indicate that disease development and treatment effects seen in our studies are not linked to the entire heterogenic pool of antigen-specific antibodies but rather to a subset with particular pathogenic or tolerogenic qualities based on specificity, affinity, or isotype, as may also be the case in MG patients.

Protein glycosylation has been found to be important in regulating immune responses, and different glycans can lead to proinflammatory or immunosuppressive signals, although the mechanisms are far from being fully understood (Front Immunol (2018) 9: 2754). Glycosylation of antigen used for therapy has been reported to play a role in i.v. tolerance induction in the case of EAE (Exp Neurol (2015) 267: 254-267). In our model of i.v. tolerance, however, protein glycosylation did not appear to play a major role, since there was no difference in the therapeutic efficacy between the P. pastoris and the E. coli expressed proteins. This was highly surprising.

A comparative study of the therapeutic efficacy of α1-ECD and pyridostigmine and methylprednisolone, two current standard of care therapeutics for MG, showed that the former had a superior effect. In fact, the clinical symptoms of rats treated with either of the two current drugs were almost identical with those of untreated animals. This highlights the qualitative difference of treatment with α1-ECD, as it had a therapeutic effect under conditions where current drugs had reduced efficacy. Furthermore, while most drugs currently used to treat MG are associated with serious long-term side effects, based on in silico immunotoxicity data α1-ECDₘ shows no risk of increased immunogenicity in humans and a preliminary toxicology study in rats showed that α1-ECDₘ was well-tolerated with no adverse clinical observations of note (data to be presented elsewhere). We, therefore, believe that our findings support the development of i.v. antigen administration as a viable therapeutic option for MG, and α 1-ECD as a potent drug candidate.

In conclusion, this example describes an effective treatment of disease in the EAMG model by intravenous delivery of a recombinant soluble major MG autoantigen, α 1-ECDₘ. α1-ECDₘ represents a promising and efficient therapeutic approach for antigen-specific treatment. In the EAMG model α1-ECDₘ shows a dose dependent capacity to induce remission after a short two-week treatment regimen. Furthermore, it provides an alternative route for clinical translation of antigen-based treatment of autoimmune diseases, as the makeup of the recombinant protein, comprising multiple auto-epitopes present in their native context, reduces the need for personalized tailoring of peptide-based treatments to overcome interindividual variability.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-28. (canceled)

29. A method of producing a fusion protein between an extracellular domain of nicotine acetylcholine receptor subunit alpha 1 (nAChRα1) and a solubility enhancing peptide, the method comprising the steps of:
solubilizing inclusion bodies comprising the fusion protein in a solubilization solution having a pH less than 11 and lacking any reducing agent to form solubilized fusion proteins;
loading the solubilized fusion proteins onto an ion-exchange resin;
eluting the loaded solubilized fusion proteins from the ion-exchange resin using an elution solution having a pH less than 11 and comprising a reducing agent to form a fusion protein eluate;
adjusting a pH of the fusion protein eluate to at least 11; and
diluting the pH-adjusted fusion protein eluate in a refolding solution having a pH of no more than 9 to form a refolded monomeric form of the fusion protein.

30. The method according to claim 29, wherein the diluting step is performed at a temperature of no more than 10° C.

31. The method according to claim 30, wherein the diluting step is performed at a temperature selected within an interval of from 4° C. to 10° C.

32. The method according to claim 29, wherein the solubilization solution has a pH selected within an interval of from 9 to 10.

33. The method according to claim 29, wherein the elution solution has a pH selected within an interval of from 9 to 10.

34. The method according to claim 29 wherein the adjusting step comprises adjusting the pH of the fusion protein eluate to within an interval of from 11 to 12.

35. The method according to claim 29, wherein the refolding solution has a pH selected within an interval of from 8 to 9.

36. The method according to claim 29, wherein the diluting step comprises diluting the pH-adjusted fusion protein eluate in the refolding solution at a volume ratio of less than 0.1:1.

37. The method according to claim 29, wherein the fusion protein comprises the extracellular domain of nAChRα1 as defined in SEQ ID NO: 3, in which amino acid residues 129 to 140 have been replaced by the solubility enhancing peptide.

38. The method according to claim 37, wherein the fusion protein comprises the extracellular domain of nAChRα1 as defined in SEQ ID NO: 3, in which amino acid residues 129 to 140 have been replaced by amino acid residues 132 to 143 from Lymnaea stagnalis acetylcholine binding-protein (AChBP) as defined in SEQ ID NO: 4.

39. The method according to claim 38, wherein the fusion protein consists of an amino acid sequence as defined in SEQ ID NO: 5 or 6.

40. The method according to claim 39, wherein the fusion protein consists of the amino acid sequence as defined in SEQ ID NO: 6.

41. The method according to claim 37, wherein the asparagine residue 141 in SEQ ID NO: 3 is not glycosylated.

42. The method according to claim 41, wherein the fusion protein has a first disulfide bond between cysteine residue 128 in SEQ ID NO: 3 and cysteine residue 142 in SEQ ID NO: 3 and a second disulfide bond between cysteine residue 192 in SEQ ID NO: 3 and cysteine residue 193 in SEQ ID NO: 3.

43. The method according to claim 29, further comprising expressing the fusion protein in bacterial cells comprising an expression vector comprising a nucleotide sequence encoding the fusion protein under control of a promoter.

44. The method according to claim 43, further comprising:
lysing the bacterial cells to form a lysate comprising the inclusion bodies; and
centrifuging the lysate to collect the inclusion bodies.

45. The method according to claim 29, further comprising purifying the refolded monomeric form of the fusion protein by ion exchange chromatography.

46. The method according to claim 45, further comprising concentrating the purified refolded monomeric form of the fusion protein by tangential flow filtration.

47. The method according to claim 46, further comprising purifying the concentrated refolded monomeric form of the fusion protein by size-exclusion chromatography.

48. An isolated fusion protein between an extracellular domain of nicotine acetylcholine receptor subunit alpha 1 (nAChRα1) as defined in SEQ ID NO: 3, in which amino acid residues 129 to 140 have been replaced by a solubility enhancing peptide, wherein the asparagine residue 141 in SEQ ID NO: 3 is not glycosylated.

49. The isolated fusion protein according to claim 48, wherein the fusion protein comprises the extracellular domain of nAChRα1 as defined in SEQ ID NO: 3, in which amino acid residues 129 to 140 have been replaced by amino acid residues 132 to 143 from Lymnaea stagnalis AChBP as defined in SEQ ID NO: 4.

50. The isolated fusion protein according to claim 49, wherein the fusion protein comprises the amino acid sequence as defined in SEQ ID NO: 5 or 6.

51. The isolated fusion protein according to claim 50, wherein the fusion protein consists of the amino acid sequence as defined in SEQ ID NO: 6.

52. The isolated fusion protein according to claim 48, wherein the fusion protein has a first disulfide bond between cysteine residue 128 in SEQ ID NO: 3 and cysteine residue 142 in SEQ ID NO: 3 and a second disulfide bond between cysteine residue 192 in SEQ ID NO: 3 and cysteine residue 193 in SEQ ID NO: 3.

53. The isolated fusion protein according to claim 48, wherein the fusion protein is in monomeric form.

54. The isolated fusion protein according to claim 48, wherein the fusion protein has a molecular weight of about 24 kDa.

55. A method for preventing, inhibiting or treating myasthenia gravis, the method comprising administering an effective amount of an isolated fusion protein according to claim 48 to a subject in need thereof.


SEQUENCE LISTING (7 sequences)

SEQ ID NO: 1 - protein, 482 aa, Homo sapiens
MEPWPLLLLFSLCSAGLVLGSEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 2 - protein, 235 aa, Homo sapiens
SEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL

SEQ ID NO: 3 - protein, 210 aa, Artificial Sequence
  Modified extracellular domain of nAChRalpha1
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL

SEQ ID NO: 4 - protein, 12 aa, Lymnaea stagnalis
DVSGVDTESGAT

SEQ ID NO: 5 - protein, 210 aa, Artificial Sequence
  TOL2 fusion protein
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRL

SEQ ID NO: 6 - protein, 211 aa, Artificial Sequence
  TOL2 fusion protein
MSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRL

SEQ ID NO: 7 - DNA, 654 bp, Artificial Sequence
  TOL2 fusion protein coding sequence
ggtctccccatgagcgaacatgaaacccgtctggttgcaaaactgttcaaagattatagcagcgttgttcgtccggttgaagatcatcgtcaggttgttgaagttaccgttggtctgcagctgatccagctgattaatgttgatgaagtgaatcagattgtgaccaccaatgttcgtctgaaacagcagtgggttgattataacctgaaatggaatccggatgattatggtggcgtgaaaaaaatccatattccgagcgaaaaaatctggcgtccggatctggttctgtataataacgcagatggtgattttgccatcgtgaaatttaccaaagttctgctgcagtataccggtcatattacctggacaccgcctgcaatctttaaaagctattgtgatgttagcggtgtggataccgaaagcggtgcaaccaattgtagcatgaaattaggcacctggacctatgatggtagcgttgttgcaattaatccggaaagcgatcagccggatctgagcaatttcatggaaagtggtgaatgggtgattaaagaaagccgtggttggaaacatagcgttacctatagctgttgtccggatacaccgtatctggatattacctatcattttgttatgcagcgcctgtaagccgagacc